(19) United States
(12) Patent Application Publication
(10) Pub. No.: US 2020/0168294 A1
(43) Pub. Date: May 28, 2020

(54) A DIAGNOSTIC AND PROGNOSTIC TEST FOR MULTIPLE CANCER TYPES BASED ON TRANSCRIPT PROFILING

(71) Applicant: UNIVERSITY OF PITTSBURGH-OF THE COMMONWEALTH SYSTEM OF HIGHER EDUCATION, Pittsburgh, PA (US)

(72) Inventors: Edward Victor PROCHOWNIK, Pittsburgh, PA (US); James Matthew DOLEZAL, Chicago, IL (US)

(21) Appl. No.: 16/631,976

(22) PCT Filed: Jul. 17, 2018
(86) PCT No.: PCT/US18/42455

§ 371 Date: Jan. 17, 2020

Related U.S. Application Data
(60) Provisional application No. 62/533,293, filed on Jul. 17, 2017.

(51) Int. Cl. G16B 25/10; G16B 40/00; G16B 20/00; G16B 45/00; G16H 50/20; G16H 70/60; G16H 70/20

(52) U.S. Cl. CPC G16B 25/10; G16B 40/00; G16B 20/00; G16H 70/20; G16H 50/20; G16H 70/60; G16B 45/00

(57) ABSTRACT

An example method of bioinformatics is described herein. The method can include receiving RNA expression data for a sample of tumor, determining a global ribosomal protein transcript (RPT) expression profile for the sample based on the RNA expression data, and identifying a tissue of origin and/or other clinical features for the sample based on the global RPT expression profile for the sample.

### CROSS-REFERENCE TO RELATED APPLICATIONS

This application claims the benefit of U.S. provisional patent application No. 62/533,293, filed on Jul. 17, 2017, and entitled “A DIAGNOSTIC AND PROGNOSTIC TEST FOR MULTIPLE CANCER TYPES BASED ON RIBOSOMAL PROTEIN TRANSCRIPT PROFILING,” the disclosure of which is expressly incorporated herein by reference in its entirety.

### STATEMENT REGARDING FEDERALLY FUNDED RESEARCH

This invention was made with government support under Grant no. CA174713 awarded by the National Institutes of Health. The government has certain rights in the invention.

### BACKGROUND

Eukaryotic ribosomes are among the most highly evolutionarily conserved organelles, comprised of four ribosomal RNAs (rRNAs) and approximately 80 ribosomal proteins (RPs). Responsible for translating mRNA into proteins, ribosomes were long believed to be nonspecific “molecular machines” with unvarying structures and function in different biological contexts. Recent evidence has shown, however, that some RPs are expressed in tissue-specific patterns and can differentially contribute to ribosome composition, affect rRNA processing, and regulate translation¹. Despite the complexity of RP assembly in ribosomes, early studies of ribosome function revealed that the catalytic activity responsible for peptide bond formation might depend only on the presence of rRNAs and a small number of core RPs². This finding, in conjunction with the observation that some RPs are expressed in a tissue-specific manner, has led some to speculate that one purpose for the evolutionary emergence of RPs may have been to confer translational specificity and adaptability to ribosomes^{1,3}.

An increasing body of evidence continues to show that RPs do, in fact, have an important role in imbuing ribosomes with mRNA translation specificity. During embryonic development, RPs are expressed at different levels across tissue types, and loss of RPs due to mutation or targeted knockdown produces specific developmental abnormalities in plants, invertebrates, and vertebrates. The tissue-specific patterning that occurs as a consequence of individual RP loss suggests that some RPs serve to guide the translation of specific subsets of transcripts in order to influence cellular development. Although the mechanism(s) by which RPs confer translation specificity are not entirely known, one may involve the alteration of ribosome affinity for transcripts with specific cis-regulatory elements, including internal ribosome entry sites (IRES) elements and upstream open reading frames (uORFs).¹

RPs also participate in a variety of extra-ribosomal functions. In normal contexts, ribosome assembly from rRNAs and RPs is a tightly regulated process, with unassembled RPs undergoing rapid degradation. Disruption of ribosomal biogenesis by any number of extracellular or intracellular stimuli induces ribosomal stress, leading to an accumulation of unincorporated RPs. These free RPs are then capable of participating in a variety of extra-ribosomal functions, including the regulation of cell cycle progression, immune signaling, and cellular development. Many free RPs bind to and inhibit MDM2, a potentially oncogenic E3 ubiquitin ligase that interacts with p53 and promotes its degradation. The resulting stabilization of p53 triggers cellular senescence or apoptosis in response to the inciting ribosomal stress. Additional extra-ribosomal functions of RPs are numerous, and have been recently reviewed^{4,5}.

Given their role in regulating gene translation, cellular differentiation, and organismal development, it is perhaps unsurprising that altered RP expression has been implicated in human pathology. Indeed, an entire class of diseases has been shown to be associated with haploinsufficient expression or mutation in individual RPs. These so-called “ribosomopathies,” including Diamond-Blackfan Anemia (DBA) and Shwachman-Diamond Syndrome (SDS), are characterized by early onset bone marrow failure, variable developmental abnormalities and a life-long cancer predisposition that commonly involves non-hematopoetic tissues^{6,7}. The loss of proper RP stoichiometry and ensuing ribosomal stress result in increased ribosome-free RPs, which bind to MDM2 and impair its ubiquitin-mediated degradation of p53^{6,8,10}. The resulting p53 stability is believed to underlie the bone marrow failure affecting erythroid or myeloid lineages in DBA and SDS, respectively. The developmental abnormalities of the ribosomopathies are variable and associate with specific RP loss or mutation. RPL5 loss in DBA, for example, is specifically associated with cleft palate and other craniofacial abnormalities whereas RPL11 loss is associated with isolated thumb malformations¹¹.

Ribosomopathy-like properties have also been observed in various cancers. It has recently been shown that RP transcripts (RPTs) were dysregulated in two murine models of hepatoblastoma and hepatocellular carcinoma in a tumor specific manner and in patterns unrelated to tumor growth rates. See Kulkarni et al., “Ribosomopathy-like Properties of Murine and Human Cancers,” PLoS ONE 12(8):e0182705, https://doi.org/10.1371/journal.pone.0182705. These murine tumors also displayed abnormal rRNA processing and increased binding of free RPs to MDM2, reminiscent of the aforementioned inherited ribosomopathies.

Perturbations of RP expression have been found in numerous human cancers, including those of the breast, pancreas, bladder, brain and many other tissues¹²⁻²⁴. Mutations and deletions of RP-encoding genes have also been found in endometrial cancer, colorectal cancer, glioma, and various hematopoietic malignancies²⁵⁻²⁷. Indeed, the Chr. 5q-abnormality associated with myelodysplastic syndrome and the accompanying haploinsufficiency of RPS14 is considered one of the prototype “acquired” ribosomopathies that are often classified together with DBA, SDS and other inherited ribosomopathies⁶. Although many free RPs can induce cellular senescence during ribosomal stress via MDM2/p53, not all RPs possess such tumor suppressor functions; RPS3A, for example, transforms NIH3T3 mouse fibroblasts and induces tumor formation in nude mice²⁸.

A recent attempt to summarize the heterogeneity of RPT expression in human cancers was limited to describing expression differences of single RPTs among cancer cohorts, without accounting for larger patterns of variation that might better distinguish tumors from one another³. RPT expression patterns were, however, examined in normal tissues using the dimensionality-reduction technique Principal Component Analysis (PCA) in the aforementioned study. These results showed hints of cell-specific patterning in the hematopoietic tissues examined, but not all cell types clustered into obviously distinct groups.

### SUMMARY

An example method of bioinformatics is described herein. The method can include receiving RNA expression data for a sample of tumor, determining a global ribosomal protein transcript (RPT) expression profile for the sample based on the RNA expression data, and identifying a tissue of origin for the sample based on the global RPT expression profile for the sample.

Additionally, the step of determining a global ribosomal protein transcript (RPT) expression profile for the sample can include calculating a respective relative expression for each of a plurality of RPTs. In some implementations, the plurality of RPTs can optionally include RPTs for approximately eighty ribosomal proteins (RPs). Alternatively or additionally, a respective relative expression can include a percentage contribution of an individual RPT to the total expression of the plurality of RPTs.

Alternatively or additionally, the step of identifying a tissue of origin for the sample can include using a classifier model. In some implementations, the classifier model can differentiate tumor tissue from normal tissue. In some implementations, the classifier model can differentiate between different types of tumor tissue. In some implementations, the classifier model can differentiate between subtypes of the same tumor tissue.

Alternatively or additionally, the method can optionally further include constructing the classifier model using respective global RPT expression profiles for a plurality of known tissues.

Alternatively or additionally, the step of identifying a tissue of origin for the sample can include comparing quantitative differences between the global RPT expression profile for the sample and one or more of the respective global RPT expression profiles for the known tissues.

Alternatively or additionally, the tissue of origin for the sample can be identified based on dysregulation of the relative expression of one or more ribosomal proteins (RPs). In some implementations, the RPs can include one or more of RPL3, RPL5, RPL8, RPL13, RPL30, RPL36, RPL38, RPL13, RPS4X, or RPS20.

Alternatively or additionally, the method can optionally further include providing a diagnosis, prognosis, or treatment recommendation based on the tissue of origin for the sample. For example, at least one of a clinical parameter, a molecular marker, or a tumor phenotype can be provided.

Alternatively or additionally, the method can optionally further include sub-classifying the tissue of origin for the sample based on the global RPT expression profile for the sample. The diagnosis, prognosis, or treatment recommendation can be provided based on a sub-class of the tissue of origin for the sample.

Alternatively or additionally, the method can optionally further include receiving the sample of tumor, extracting RNA from the sample, isolating a plurality of RPTs from the extracted RNA, and obtaining the RNA expression data from the isolated RPTs.

Alternatively or additionally, in some implementations, the RNA expression data can include RNA-seq data. Alternatively or additionally, in some implementations, the RNA expression data can include microarray data.

Alternatively or additionally, the method can optionally further include receiving respective RNA expression data and respective clinical information for each of a plurality of tumors from a database, determining respective global RPT expression profiles for the tumors in the database based on the respective RNA expression data, identifying recurring patterns of RPT expression among the tumors in the database, and comparing the recurring patterns of RPT expression with the respective clinical parameters.

Alternatively or additionally, in some implementations, the step of identifying a tissue of origin for the sample can include comparing the global RPT expression profile for the sample to the respective global RPT expression profiles for the tumors in the database.

Alternatively or additionally, in some implementations, the step of identifying recurring patterns of RPT expression among tumors in the database can include applying a machine learning model that analyzes linear and non-linear relationships among the respective relative expression for each of the plurality of RPTs. Optionally, the machine learning model can be t-distributed stochastic neighbor embedding (t-SNE).

Alternatively or additionally, the method can further include graphically displaying the global RPT expression pattern for the sample with clusters using a three-dimensional (3D) map.

Another method of bioinformatics is described herein. The method can include determining a global ribosomal protein transcript (RPT) expression profile for a sample of tumor, and identifying a tissue of origin for the sample based on the global RPT expression pattern for the sample.

Yet another method of bioinformatics is described herein. The method can include receiving RNA expression data for a sample of tumor, determining a global ribosomal protein transcript (RPT) expression profile for the sample based on the RNA expression data, and providing a diagnosis, prognosis, or treatment recommendation based on the global RPT expression profile.

Yet another example method of bioinformatics is described herein. The method can include receiving RNA expression data for a sample of tumor, determining a global cholesterol biosynthesis transcript expression profile for the sample based on the RNA expression data, and providing a diagnosis, prognosis, or treatment recommendation based on the cholesterol biosynthesis transcript expression profile.

Yet another example method of bioinformatics is described herein. The method can include receiving RNA expression data for a sample of tumor, determining a global fatty acid oxidation (FAO) transcript expression profile for the sample based on the RNA expression data, and providing a diagnosis, prognosis, or treatment recommendation based on the FAO transcript expression profile.

Yet another example method of bioinformatics is described herein. The method can include receiving RNA expression data for a sample of tumor, determining a global transcript expression profile for the sample based on the RNA expression data, and providing a diagnosis, prognosis, or treatment recommendation based on the transcript expression profile. The step of determining a global transcript expression profile for the sample can include calculating a respective relative expression for each of a plurality of transcripts. Additionally, a machine learning algorithm that is configured to analyze linear and non-linear relationships in a dataset can be used to identify patterns of transcript expression. Optionally, the machine learning algorithm can be t-SNE.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a flow chart illustrating an example method of bioinformatics according to implementations described herein.

FIG. 2 is an example computing device.

FIGS. 3A-3E illustrate how t-SNE better identifies clusters of RPT expression as compared to PCA. FIG. 3A illustrates relative expression of RPTs in normal tissues from five cohorts was analyzed with PCA. In both methods, clustering occurs when samples possess similar underlying patterns of variation. t-SNE provides more distinct clusters that better associate with tissue of origin, indicating that normal tissues have distinct patterns of RPT expression. Axes are not labeled with t-SNE, as points are not mapped linearly and axes are not directly interpretable. FIG. 3B illustrates similar analyses to those of FIG. 3A in tumors. PCA clusters are poorly defined and do not correlate strongly with tumor type. t-SNE clusters are distinct and strongly associate with cancer type, indicating that tumors possess unique patterns of RPT expression based on their tissue of origin. FIG. 3C illustrates combined t-SNE analysis of RPT expression in normal tissue and tumor samples. Normal tissues and tumors cluster together but can be distinguished from one another, indicating that the latter retain a pattern of RPT expression resembling that of the normal tissue from which they originated. FIG. 3D illustrates many single cancer cohorts demonstrate sub-clustering by t-SNE. Clustering of six cohorts are provided as examples here. The number of clusters found in each cohort is listed in Supplementary Table 1 shown in FIG. 14. FIG. 3E illustrates 3D area map of RPT relative expression in tumors from two cancer cohorts, sorted by cluster. The x-axis represents individual tumors, the z-axis represents individual RPTs, and the y-axis represents deviation from the mean relative expression. Cluster 2 of prostate cancer and Cluster 3 of HCC are both comprised of tumors with high relative expression of RPL8 and low RPL3. FIGS. 9B, 10, and 13 illustrate additional t-SNE plots of tumors and normal tissues. Perplexity settings for t-SNE analyses are designated in each plot by “P:”. For all analyses, learning rate (epsilon)=10 and iterations=5000.

FIG. 4 illustrates volcano plots of relative RPT expression in tumor clusters in twelve cancer cohorts. Relative expression of RPTs was compared between tumor clusters in each included cancer cohort with ANOVA tests. The negative log of the ANOVA P-value for each RPT is displayed on the y-axis and the difference in relative expression across tumor clusters is displayed on the x-axis. RPTs near the top of the graphs are most significantly differentially expressed between tumor clusters. Note that nearly every RPT in virtually all cancer cohorts falls above −log(P) of 2, corresponding to P<0.01 and indicating that tumor clusters have significantly distinct expression of virtually all RPTs. For each cohort, the number of samples in each cluster are shown under the label “n”. Additional volcano plots of seven other cancer cohorts are continued in FIG. 5A. In FIG. 4, the tumor cohorts are labelled large B-cell lymphoma (DLBC), head and neck (HNSC), kidney chromophobe (KICH), acute myeloid leukemia (LAML), lung (LUNG), pancreatic (PAAD), pheochromocytoma and paraganglioma (PCPG), prostate (PRAD), stomach (STAD), testicular (TGCT), thyroid carcinoma (THCA), and thymoma (THYM).

FIGS. 5A-5B illustrate volcano plots of relative RPT expression in tumor clusters associated with survival. FIG. 5A illustrates volcano plots comparing RPT relative expression between tumor clusters were generated, as in FIG. 4, for the remaining seven cancer cohorts which possessed tumor sub-clustering by t-SNE. Note that for the sake of clarity, clusters 5 and 6 are excluded from the LUNG cohort plot. These clusters correlated near perfectly with amplification and highly significant up-regulation of RPS3 and RPS16, respectively (Table 2 shown in FIG. 7). FIG. 5B illustrates patient survival by t-SNE cluster. Of the 19 cancer cohorts with sub-clustering of RPT expression patterns by t-SNE, seven possessed clusters that correlated with survival. Significance was determined with log-rank and Wilcoxon rank sum tests where appropriate, using all survival data available, including any data points beyond what are displayed in the survival curves. In FIG. 5A, the tumor cohorts are labelled breast (BRCA), liver (LIHC), uterine corpus endometrial carcinoma (UCEC), kidney clear cell carcinoma (KIRC), melanoma (SKCM), cervical (CESC), and glioblastoma multiforme and low-grade glioma (GBMLGG).

FIG. 6 includes Table 1, which shows recurring patterns of RPT relative expression across cancer cohorts. Certain patterns of expression distinguishing tumor clusters from one another were observed in multiple clusters across cancer cohorts, as shown in FIG. 4 and FIG. 5A. In this table, “low” refers to tumor clusters expressing lower relative expression of a given RPT relative to other tumors in the given cancer cohort, and “high” refers to clusters with greater relative expression compared to other tumors.

FIG. 7 includes Table 2, which shows RP gene copy number alterations associated with t-SNE clusters. Some tumor clusters were significantly associated with greater incidence of copy number alterations than other tumors from the same cancer cohorts (α<0.01); clusters with >90% of tumors possessing a given copy number alteration are included in this table.

FIG. 8 includes Table 3, which shows tumor phenotypes and clinical parameters associated with t-SNE clustering. Tumor phenotypes and clinical markers were compared between tumor clusters using Chi-squared tests, with significance defined as α<0.01. “Other tumors” are comprised of all tumors from the same cancer cohort not falling into the given cluster. Data were obtained using the Xena Functional Genomics Explorer from the University of California Santa Cruz, https://xenabrowser.net (referred to herein as the “UCSC Xenabrowser”), under the data heading “Phenotypes.”

FIGS. 9A-9B illustrate normal tissues cluster distinctly with t-SNE. RPT expression in normal tissue samples from cohorts with at least 10 normal tissues was visualized with two dimensionality reduction techniques, PCA (shown in FIG. 9A) and t-SNE (shown in FIG. 9B). Using PCA, normal tissue samples exhibit slight clustering according to tissue type, but differences in RPT expression between cohorts are not distinct. With t-SNE, normal tissues cluster according to tissue type nearly perfectly. Note that overlap occurs between samples from kidney chromophobe (KICH), kidney clear cell carcinoma (KIRC) and kidney papillary cell carcinoma (KIRP) due to the fact that normal tissues are all kidney in these cohorts. The esophageal cancer cohort¹⁵ was excluded from this graph, as data were missing expression of five RPTs—RPL17, RPL36A, RPS10, RPS17, and RPS4Y1. Parameters used for t-SNE: perplexity=31, learning rate=10, iterations=5000.

FIG. 10 illustrates normal tissues cluster distinctly from tumors of the same tissue type. RPT expression of both normal tissue and tumor samples were analyzed with t-SNE in all cohorts with at least 10 normal tissue samples. Tumors are colored black, and normal tissues are colored gray. Normal tissues sub-cluster together distinctly from tumors but within the larger tumor cluster. Thus, RPT expression in tumors is similar to, but distinct from, normal tissues, and tumors have greater overall heterogeneity in their RPT expression patterns. t-SNE parameters for all plots: perplexity=60, learning rate=10, iterations=2000. In FIG. 10, the tumor cohorts are labelled bladder (BLCA), breast (BRCA), colorectal (COADREAD), esophageal carcinoma (ESCA), head and neck (HNSC), kidney chromophobe (KICH), kidney clear cell carcinoma (KIRC), kidney papillary cell carcinoma (KIRP), liver (LIHC), lung (LUNG), prostate (PRAD), stomach (STAD), thyroid carcinoma (THCA), and uterine corpus endometrial carcinoma (UCEC).

FIG. 11 illustrates tumor cohorts with overlapping RPT expression profiles. Five cancer cohorts were comprised of tumors with overlapping RPT expression patterns and did not cluster distinctly with t-SNE. These cohorts—cholangiocarcinoma (CHOL), lung (LUNG), bladder (BLCA), cervical (CESC), and uterine carcinosarcoma (UCS)—were grouped together, here referred to as “mixed cancers.” This group of mixed cancers displayed significant overlap with five other cohorts that otherwise clustered with fair distinction from one another—colorectal (COADREAD), liver (LIHC), mesothelioma (MESO), pancreatic (PAAD), and skin cutaneous melanoma (SKCM). These five cohorts were analyzed alongside the mixed cancer group with t-SNE with the results shown here. The following t-SNE parameters were used: perplexity=24, learning rate (epsilon)=10, iterations=5000.

FIG. 12 illustrates pan-cancer t-SNE plot reveals tumor clusters not associating with tissue of origin. Three-dimensional t-SNE analysis of RPT expression in tumors from 29 cancer cohorts. Tumors from ESCA were excluded from this pan-cancer analysis due to the missing expression of five RPTs: RPL17, RPL36A, RPS10, RPS17, and RPS4Y1. In addition to the numerous clusters associated with tumor type, two clusters were identified that did not associate with tissue of origin. Both are circled in FIG. 12. The first, labeled 1202, was comprised of 143 tumors, all of which shared relative up-regulation of RPL19 and RPL23, along with amplification of a region on 17q12 containing the genes RPL19, RPL23, and ERBB2 (Her2/Neu). These tumors were from the following cohorts: BLCA, BRCA, CESC, COADREAD, HNSC, LUNG, PAAD, SKCM, STAD, KIRC, KIRP, OV, THYM, UCEC, and UCS. The second cluster, labeled 1204, was comprised of 77 tumors, and no discernable shared RPT expression pattern could be identified in this group. These tumors were from the cohorts BLCA, BRCA, CESC, COADREAD, HNSC, LUNG, OV, PAAD, SARC, SKCM, TGCT, and UCS.

FIG. 13 illustrates sub-clustering of RPT expression patterns in additional tumor cohorts. t-SNE plots of tumor RPT expression patterns in 13 cohorts with sub-clusters, in addition to those already displayed in FIG. 3D. Perplexity settings for t-SNE analyses are designated in each plot by “P:”. All analyses were performed with learning rate (epsilon)=10 and iterations=5000.

FIG. 14 includes Supplementary Table 1, which shows the Cancer Genome Atlas (TCGA) cohorts and clusters identified by t-SNE. Relative expression of RPTs was calculated using RNA-seq expression data from TCGA, accessed via the UCSC Xenabrowser. Clustering of RPT expression was investigated with t-SNE using TENSORFLOW, which is open-source software developed by GOOGLE, INC. of Mountain View, Calif., with perplexity varying between 6-15. Exact parameters used for final t-SNE plots can be found in the respective figures (FIG. 3D and FIG. 13). Clusters were defined as groups of >10 tumors visually separating into distinct clusters (FIG. 4). Nineteen cancer cohorts demonstrated distinct clustering by t-SNE. Cancer cohorts without sub-clustering are denoted with “-”.

FIG. 15 includes Supplementary Table 2, which shows logistic regression (LR) and Artificial Neural Network (ANN) models classify tumors by RPT expression. Using RPT expression, various models were constructed to predict features identified by the previous t-SNE analyses. ANNs were constructed with TENSORFLOW, which is open-source software developed by GOOGLE, INC. of Mountain View, Calif., and trained on 60% of data, with 10% of data saved for validation during hyper-parameter tuning. For ANNs, “accuracy” reflects classification accuracy of the final chosen model after hyper-parameter tuning on a separate test set, comprised of 30% of the original data. All data for ANN training and testing was balanced by cancer cohort to reduce the risk of bias, such that the same number of samples from each cohort were included in training and testing. LR models were constructed using Stata SE.

FIG. 16 is a flow chart illustrating another example method of bioinformatics according to implementations described herein.

FIGS. 17A-17G illustrate the results of analyses performed on transcripts involved in cholesterol biosynthesis, fatty acid oxidation (FAO) synthesis, and glycolysis. FIG. 17A illustrates mean expression levels of cholesterol biosynthetic enzyme-encoding transcripts for 371 human HCC samples and 50 matched liver samples. FIG. 17B illustrates the survival of patients whose tumors expressed the highest and lowest levels of the transcripts shown in FIG. 17A. FIG. 17C illustrates differences in cholesterol biosynthesis transcript expression of the transcripts shown in FIG. 17A. FIG. 17D illustrates three distinct HCC groups identified as a result of performing the t-SNE analysis. FIG. 17E illustrates the survival of patients diagnosed with each of the three distinct HCC groups shown in FIG. 17D. FIG. 17F illustrates FAO:glycolytic transcript ratios. FIG. 17G compares the survival of patients with FAO:glycolytic transcript ratios in the highest and lowest quadrants.

FIGS. 18A-18B illustrate expression of transcripts encoding enzymes involved in cholesterol biosynthesis. FIG. 18A illustrates the pathway of cholesterol biosynthesis. Enzymes whose respective transcripts were used for the construction of heat maps, are indicated in gray. FIG. 18B illustrates heat map of cholesterol biosynthesis transcript expression. The depicted heat map includes mean expression values for each transcript based on RNAseq profiling from five animals/group.

FIGS. 19A-19C illustrate expression of transcripts encoding proteins involved in fatty acid (FA) metabolism. FIG. 19A illustrates the heat map for fatty acid synthesis transcripts including mean expression values based on RNAseq profiling. FIG. 19B illustrates pathway for FAO. Enzymes whose respective transcripts were used for the construction of heat maps, are indicated in gray. FIG. 19C illustrates heat map of FAO transcript expression. The heat map includes mean expression values.

FIG. 20 illustrates t-SNE analysis of cholesterol biosynthetic transcript patterns identifies distinct tumor groups that correlate with patient survival. t-SNE patterns for the transcripts were calculated from TCGA expression profiles and displayed as described herein. Where available, t-SNE patterns for matched normal human tissues were similarly calculated and plotted. Survival data for each of the tumor cohorts were then plotted as shown in FIG. 17G.

FIG. 21 illustrates random Forest classification of cholesterol biosynthesis-related transcripts most responsible for t-SNE clustering. Each of the histograms indicates the transcripts most deterministic of the patterns depicted in FIG. 20.

FIG. 22 illustrates distribution of FAO- and glycolysis-related transcripts and Kaplan-Meier survival curves as depicted in FIGS. 17F and 17G for seven other human cancers. Data from TCGA were analyzed as described herein.

FIG. 23 illustrates t-SNE analysis of FAO-related transcripts identifies distinct tumor groups that correlate with patient survival. t-SNE for the FAO transcripts were analyzed in the same 32 TCGA tumor types used to construct the cholesterol transcript t-SNE expression profiles shown in FIG. 20. Kaplan-Meier survival curves were then plotted for each of the clusters.

FIG. 24 illustrates random Forest classification of FAO-related transcripts most responsible for t-SNE clustering. Each of the histograms indicates those transcripts which were the most deterministic of the patterns depicted in FIG. 23.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As described above, ribosomes, the organelles responsible for the translation of mRNA, are comprised of rRNA and approximately 80 RPs. Although canonically assumed to be maintained in equivalent proportions, some RPs have been shown to possess differential expression across tissue types. Dysregulation of RP expression occurs in a variety of human diseases, notably in many cancers, and altered expression of some RPs correlates with different tumor phenotypes and patient survival. To investigate the impact of global RP transcript (RPT) expression patterns on tumor phenotypes, RPT expression of ˜10,000 human tumors and 700 normal tissues were analyzed with t-distributed stochastic neighbor embedding (t-SNE). As described herein, normal tissues and cancers are shown to possess readily discernible RPT expression patterns. In tumors, this patterning is distinct from normal tissues, distinguishes tumor subtypes from one another, and in many cases correlates with molecular, pathological, and clinical features, including survival. Collectively, RPT expression can be used as a method of tumor classification, offering a potential clinical tool for prognosis and therapeutic stratification.

As described below, a machine learning technique known as t-SNE is used to identify distinct patterns of RPT expression across both normal human tissues and cancers. Like PCA, t-SNE is a dimensionality reduction technique used to visualize patterns in a data set²⁹. With either technique, patterns shared between data points are represented with clustering. t-SNE differs from PCA in that it performs particularly well with highly dimensional data and is able to distinguish non-linear relationships and patterns. With t-SNE, virtually all normal tissues and tumors can be reliably distinguished from one another based on their RPT expression profile. Tumors are readily distinguishable from normal tissues, but retain sufficient normal tissue patterning to allow for their origin to be easily discerned. Finally, a number of cancers possess subtypes of RPT expression patterns that correlate in readily understandable ways with molecular markers, various tumor phenotypes, and survival.

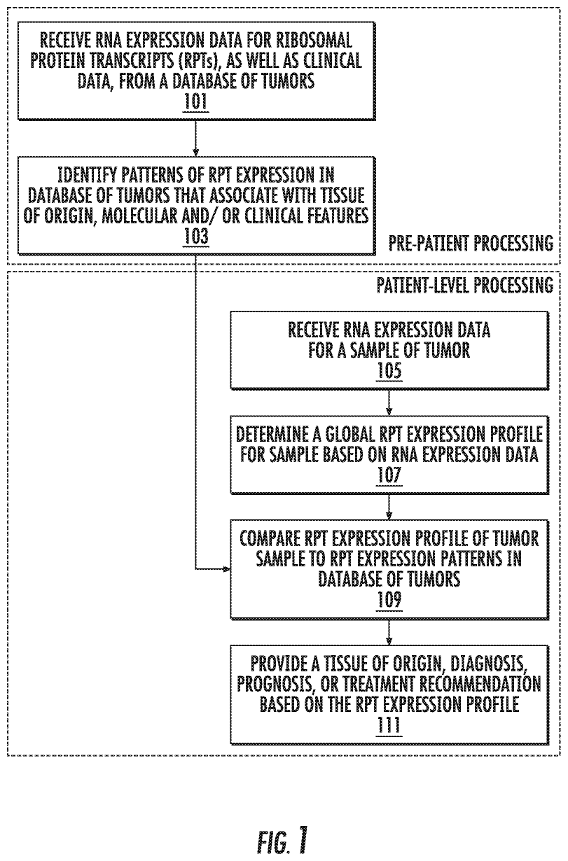

Referring now to FIG. 1, a flow chart illustrating example operations for a bioinformatics method described herein is shown. FIG. 1 illustrates pre-patient processing steps (e.g., steps 101 and 103) and patient-level processing steps (e.g., steps 105-111). At 101, a database of RNA expression data that includes expression of RPTs (e.g., RNA-seq, whole transcriptome sequence data, or microarray data) for a plurality of tumors is received or accessed. Optionally, clinical data for the patients from which these tumors derive can also be received or accessed at step 101. Such a database can include, but is not limited to, The Cancer Genome Atlas (TCGA). At 105, RNA expression data that includes the expression of RPTs for a sample of tumor (sometimes referred to herein as “individual tumor sample”) is received. The tissue of origin of this tumor may be known or unknown (e.g., an undifferentiated tumor). For example, a tissue sample from a tumor in a subject's organ (e.g., liver) is taken by a surgeon. The tissue sample can be taken, for example, by performing a biopsy. An examination of the cells in this sample by a pathologist may not reveal in which of the subject's organs (e.g., colon, pancreas, ovary, etc.) the cancer arises because the cells may appear immature and/or primitive and therefore difficult to identify. It should be understood that the tissue of origin is relevant to diagnosis, prognosis, and/or treatment. For example, not only are ovarian colo-rectal and pancreatic cancers treated very differently but they have vastly different survival.

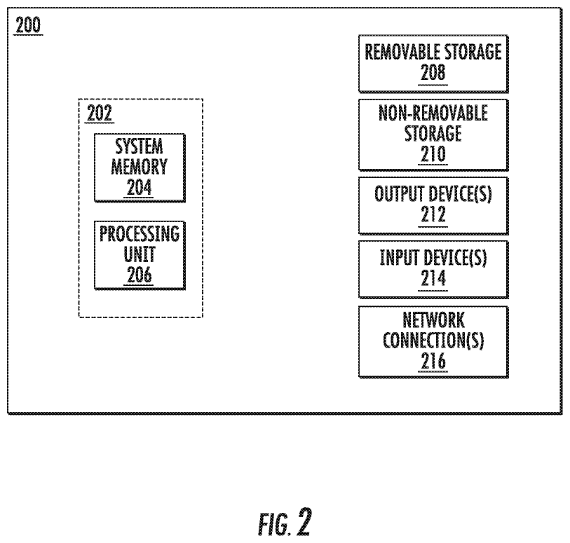

In some implementations, the RNA expression data for the individual tumor sample is received, for example, at a computing device (e.g., computing device 200 of FIG. 2). In other implementations, the sample of tumor is optionally received, for example, at a laboratory or other facility for analysis. In this case, the method can include extracting RNA from the sample and isolating RPTs from the same. After isolating the RPTs, the RP RNA expression data can be obtained by sequencing the same. This disclosure contemplates providing a kit for facilitating extraction of RNA from the sample and isolation of the RPTs. Techniques for extracting RNA, isolating RNAs, and sequencing are known in the art. Additionally, techniques for specifically isolating RPTs are similar to techniques that have been used for other transcripts. For example, in some implementations, magnetic beads with oligonucleotides corresponding to the compliment of the coding sequence of the RPTs can be used to isolate the RPTs. It should be understood that this is only one example technique for isolating the RPTs and that other techniques can be used with the bioinformatics methods described herein. Additionally, this disclosure contemplates obtaining RNA expression data using other techniques including, but not limited to, using microarray- or hybridization-based systems. For example, it should be understood that the ribosomal protein transcript (RPT) expression pattern for a sample can be determined using a DNA microarray. DNA microarrays are known in the art and are therefore not described in further detail herein. Accordingly, the RNA expression data can be of any type and in some embodiments comprises whole or partial transcriptome sequence data (e.g., RNA-seq), RP sequence data, and/or microarray hybridization data.

At 103, global ribosomal protein transcript (RPT) expression patterns or profiles for tumors in the database are determined based on the RNA expression data for the tumors received at step 101. At 107, a global RPT expression profile for the individual tumor sample is determined based on the RNA expression data received at step 105. This disclosure contemplates that the global RPT expression patterns or profiles can be determined using a computing device (e.g., computing device 200 of FIG. 2). This can include a pre-processing step of calculating a respective relative expression for each of a plurality of RPTs. Pre-processing is performed on the raw RNA expression data received at steps 101 (for the database of tumors) and 105 (for the individual tumor sample). As described herein, the plurality of RPTs can include RPTs for approximately eighty ribosomal proteins (RPs). Additionally, a respective relative expression can be defined as a percentage contribution of an individual RPT to the total expression of the plurality of RPTs. After calculating the respective relative expression for each of a plurality of RPTs, a machine learning model is used to identify patterns of RPT relative expression in the database of tumors while analyzing linear and non-linear relationships among the respective relative expression for each of the plurality of RPTs. As described herein, the machine learning model can optionally be t-distributed stochastic neighbor embedding (t-SNE). t-SNE has advantages as compared to data analysis techniques such as PCA, particularly because t-SNE is able to identify common patterns and features in a data set while accounting for both linear and non-linear relationships It should be understood that t-SNE is only one example machine learning model. This disclosure contemplates that other machine learning models can be used with the bioinformatics methods described herein. Patterns of RPT expression in the tumors from the database which have been identified by a machine learning model can be compared to clinical information about the patients from which these tumors derive with standard statistical tests. Such statistical tests can include, but are not limited to, t-tests, Chi-square tests, and/or log-rank tests. Such clinical information can include, but is not limited to, tumor type, patient survival, treatment response, or tumor biomarkers. Patterns of RPT expression that significantly associate with clinical parameters can be identified. At 109, the global RPT expression profile from the individual tumor sample can be compared to the aforementioned RPT expression patterns identified in the database. Optionally, as described herein, global RPT expression for the tumors in the database, as well the individual tumor sample, can be graphically displayed with clusters using a three-dimensional (3D) map. It should be understood that this allows the user to visualize patterns in the data set.

At 111 a tissue of origin, diagnosis, prognosis, or treatment recommendation is provided based on the comparison between the global RPT expression profile of the individual tumor sample and the RPT expression patterns identified in the database. For example, at least one of a clinical parameter (e.g., survivability metric), a molecular marker, or a tumor phenotype can be provided. As described herein, in some implementations, the tissue of origin for the sample can be sub-classified based on the global RPT expression pattern for the sample. The sub-classification can then be used when providing the diagnosis, prognosis, or treatment recommendation. This disclosure contemplates that any of the aforementioned information can be provided using a computing device (e.g., computing device 200 of FIG. 2). The comparison between the individual patient sample and the database of tumors is performed with the use of a classifier model. As described herein, a classifier model can be used to identify the tissue of origin for the sample, histologic subtype, prognostic group, or other clinical parameters. In some implementations, the classifier model is an artificial neural network (ANN) or a logistic regression (LR) classifier. It should be understood that ANN and LR classifiers are only example classifier models. This disclosure contemplates that other classifier models can be used with the bioinformatics methods described herein. The classifier model can differentiate tumor tissue from normal tissue. Alternatively or additionally, the classifier model can differentiate between different types of tumor tissue. Alternatively or additionally, the classifier model can differentiate between subtypes of the same tumor tissue (i.e., sub-classify a particular type of tumor). In other words, using the global RPT expression pattern for the sample, it is possible (e.g., by comparison with a data set) to identify the tissue of origin. As described herein, both normal and tumor tissues normal tissues possess readily discernible RPT expression patterns. One advantage of the neural network classifier is that its reliability and predictability become progressively better as it “learns” to classify different tumors types and distinguish their RPT expression patterns from those of normal tissues.

As described herein, the classifier model can be constructed using respective global RPT expression patterns for a plurality of known tissues (e.g., a majority of known tissues). As discussed above, when using a neural network, reliability and predictability improve when trained with more data. For example, global RPT expression patterns can be obtained by pre-processing raw RNA-seq expression data and applying a machine learning model (e.g., t-SNE) as described above. RNA-seq expression data for known tissue can be obtained from databases including, but not limited to, The Cancer Genome Atlas (TCGA). The global RPT expression patterns for known tissues can be used to train the classifier model. It should be understood that such training improves performance of the classifier model. In some implementations, the tissue of origin can be identified by comparing quantitative differences (e.g., statistical differences such as Analysis of Variation (ANOVA)) between the global RPT expression pattern for the sample and one or more of the respective global RPT expression patterns for the known tissues. Alternatively or additionally, it is possible to graphically display (e.g., by generating volcano plots comparing RPT expression patterns) one or more of the global RPT expression patterns, which can provide a visual indication of patterns in the data set, to identify the tissue of origin.

The techniques described above with regard to FIG. 1 leverage patterns of global RPT expression to distinguish normal tissue from tumor tissue with a higher degree of reliability and confidence as compared to conventional techniques. Alternatively or additionally, the techniques described above with regard to FIG. 1 leverage patterns of global RPT expression to categorize tumors into subtypes that were previously unrecognized with conventional techniques. This is made possible, in part, by applying a machine learning model capable of analyzing linear and non-linear relationships (e.g., t-SNE) in data. Further, as described herein, the global RPT expression patterns can be correlated with clinical parameters, molecular markers, cancer phenotypes, and/or survivability. It should be understood that such information can be used to diagnose and/or treat a disease.

It should be appreciated that the logical operations described herein with respect to the various figures may be implemented (1) as a sequence of computer implemented acts or program modules (i.e., software) running on a computing device (e.g., the computing device described in FIG. 2), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 2, an example computing device 200 upon which embodiments of the invention may be implemented is illustrated. It should be understood that the example computing device 200 is only one example of a suitable computing environment upon which embodiments of the invention may be implemented. Optionally, the computing device 200 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 200 typically includes at least one processing unit 206 and system memory 204. Depending on the exact configuration and type of computing device, system memory 204 may be volatile (such as random access memory (RAM)), non-volatile (such as read-only memory (ROM), flash memory, etc.), or some combination of the two. This most basic configuration is illustrated in FIG. 2 by dashed line 202. The processing unit 206 may be a standard programmable processor that performs arithmetic and logic operations necessary for operation of the computing device 200. The computing device 200 may also include a bus or other communication mechanism for communicating information among various components of the computing device 200.

Computing device 200 may have additional features/functionality. For example, computing device 200 may include additional storage such as removable storage 208 and non-removable storage 210 including, but not limited to, magnetic or optical disks or tapes. Computing device 200 may also contain network connection(s) 216 that allow the device to communicate with other devices. Computing device 200 may also have input device(s) 214 such as a keyboard, mouse, touch screen, etc. Output device(s) 212 such as a display, speakers, printer, etc. may also be included. The additional devices may be connected to the bus in order to facilitate communication of data among the components of the computing device 200. All these devices are well known in the art and need not be discussed at length here.

The processing unit 206 may be configured to execute program code encoded in tangible, computer-readable media. Tangible, computer-readable media refers to any media that is capable of providing data that causes the computing device 200 (i.e., a machine) to operate in a particular fashion. Various computer-readable media may be utilized to provide instructions to the processing unit 206 for execution. Example tangible, computer-readable media may include, but is not limited to, volatile media, non-volatile media, removable media and non-removable media implemented in any method or technology for storage of information such as computer readable instructions, data structures, program modules or other data. System memory 204, removable storage 208, and non-removable storage 210 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 206 may execute program code stored in the system memory 204. For example, the bus may carry data to the system memory 204, from which the processing unit 206 receives and executes instructions. The data received by the system memory 204 may optionally be stored on the removable storage 208 or the non-removable storage 210 before or after execution by the processing unit 206.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### Examples

It has been known for many years that tumors up-regulate protein biosynthesis in order to maintain their rapid growth. Coincident with this, tumors increase the levels of transcripts for each of the approximately 80 RPs that comprise the 40S and 605 subunits of the mature 80S ribosome. It was recently discovered that, in addition to the up-regulation of RP transcripts in two models of human liver cancer (hepatoblastoma [HB] and hepatocellular carcinoma [HCC]), these tumors also alter the relative degree to which each of the transcripts is up-regulated such that the pattern of expression in livers and tumors is distinctly different and predictable. The abnormal pattern of RP transcript dysregulation is reminiscent of a category of mostly pediatric hematologic disorders known as the ribosomopathies in which mutational inactivation leads to haploinsufficiency of one of about a dozen RPs leading to bone marrow failure, growth defects and a cancer predisposition. Indeed, the pattern of RP transcript dysregulation in murine HBs and HCCs appeared to represent a grossly exaggerated form of ribosomopathy. It has also been shown that several other features of ribosomopathies are present in these tumors, including the ability to efficiently process rRNA precursors. Thus, human cancers may in fact be a common and highly exaggerated manifestation of what had previously been thought to be an otherwise obscure and uncommon set of pediatric hematologic disorders.

The above observations in experimental murine tumors raised the question as to whether RP transcript dysregulation of a similar magnitude could be observed in naturally-occurring human cancers. To this end, publically available transcriptome profile results were queried from the cancer genome atlas (TCGA) (https://cancergenome.nih.gov) for ˜10,000 tumors comprising ˜30 different human cancer types and their corresponding normal tissues and then applied an advanced form of machine learning termed t-SNE to identify and classify RP transcript patterns based on a variety of linear and non-linear relationships. This is a much more powerful means of representing high-complexity data sets than techniques such as PCA which analyze linear relationships only. When examined in this way, the following observations were made: 1. All normal tissues can be distinguished from one another based simply on the patterns of their RP transcript expression; 2. All tumors can also be distinguished from one another; 3. RP transcript profiles of tumors and the normal tissues from which they arise bear a close relationship to one another but can readily be discerned with >95% accuracy; 4. In at least ten different common tumors types, including HCC, kidney, brain and endometrial cancer, the severe and/or pattern of RP transcript dysregulation is highly predictive of survival; 5. Within certain cancer groups, RP transcript profiling reveals the presence of two or more subtypes that correlate with already known clinical parameters. For example Her2+ and Her2− breast cancers can be readily distinguished as can glioblastoma multiforme, astrocytoma and non-astrocytic low-gradegliomas in the case of brain tumors.

Taken together, the above results suggest that RP transcript profiling, combined with a t-SNE-based analysis program can potentially be developed into a clinically useful bioinformatics platform to: 1. determine the tissue of origin of certain types of undifferentiated tumors such that the most appropriate therapeutic options can be selected for individual patients; 2. more accurately classify known tumors into clinically important subtypes; and 3. stratify patients with heretofore indistinguishable tumors into high and low-risk categories.

Molecular profiling of certain tumors such as breast cancer is already being used routinely in clinical practice. For example the MammaPrint test (Agendia Corp) is molecular diagnostic test based on the expression of about 70 genes in early stage breast cancer patients. It predicts the likelihood that a tumor will metastasize such that patients with low scores can safely forego chemotherapy without decreasing the likelihood of disease free survival. However, its major shortcoming is that it is useful only for early stage breast cancer. The advantage of RP transcript profiling is that, unlike the MammaPrint test, the same of group of RP genes can potentially be used for prognosis and treatment decisions across multiple cancer types and subtypes.

Results

t-SNE Identifies Tissue- and Tumor-Specific RPT Expression

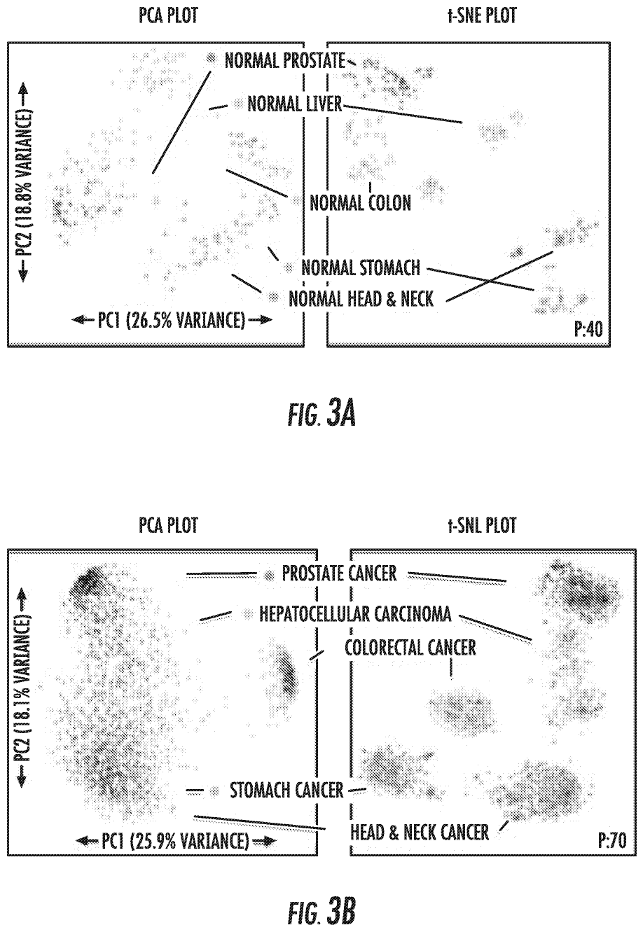

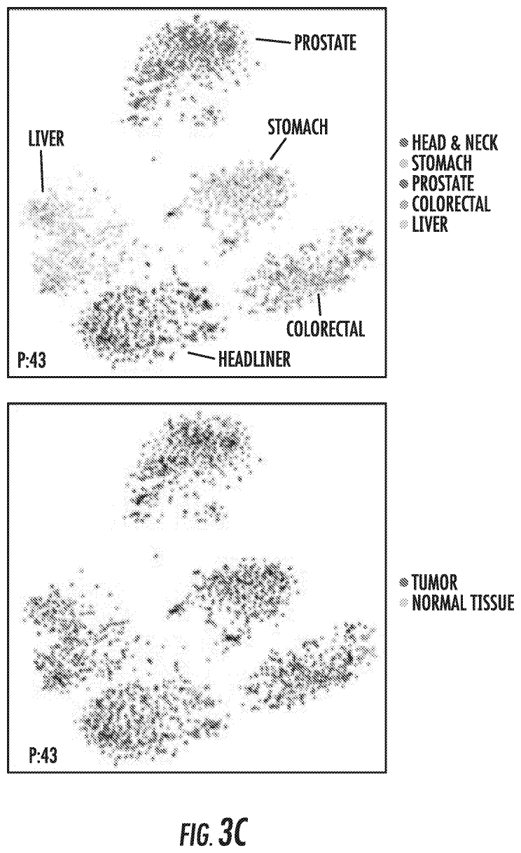

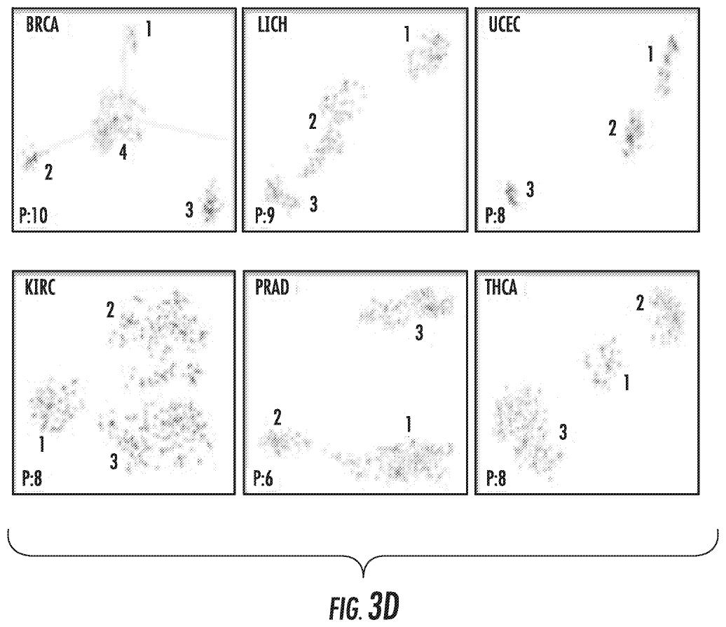

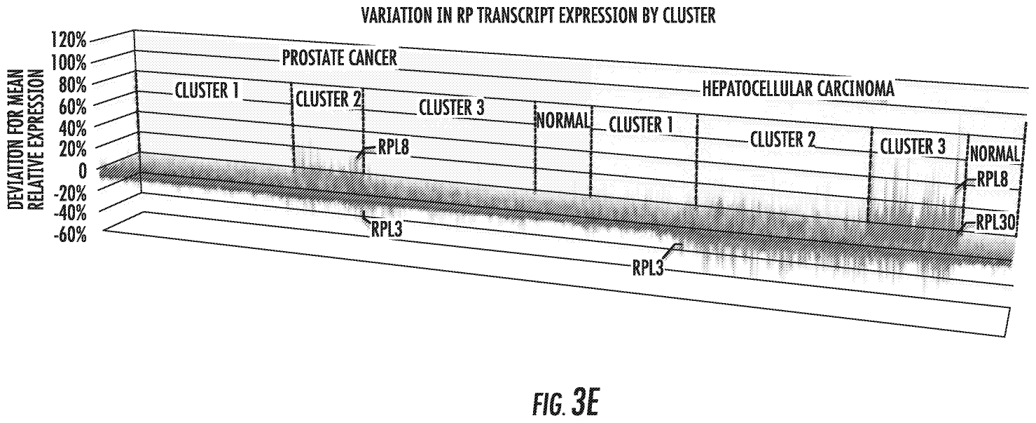

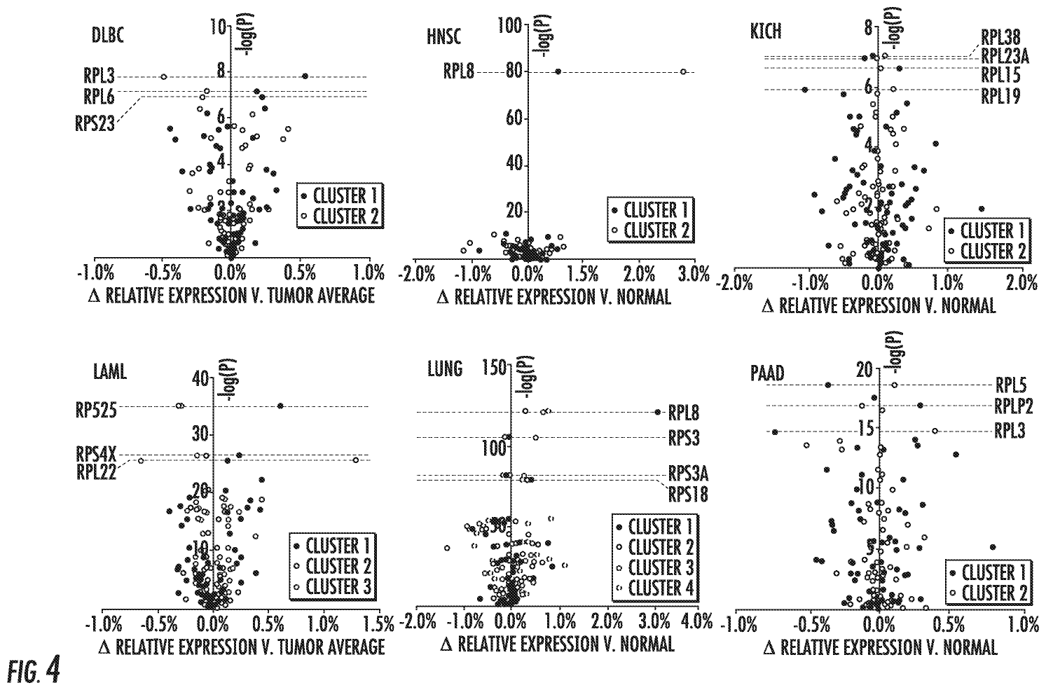

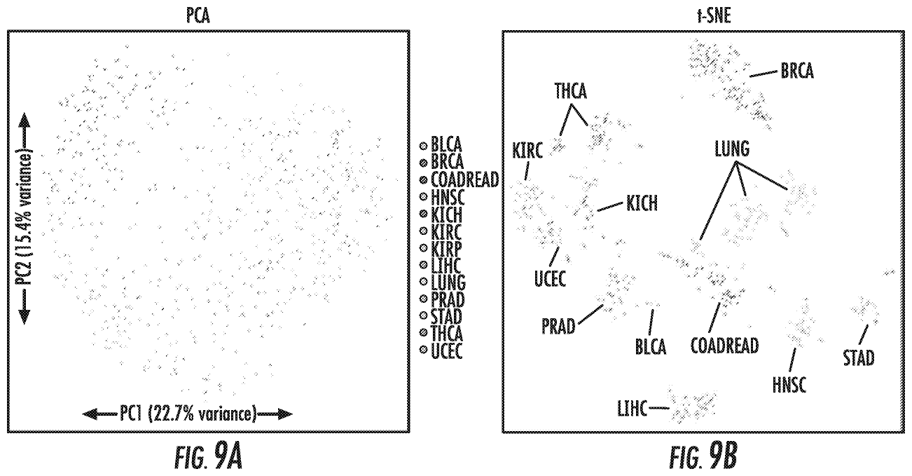

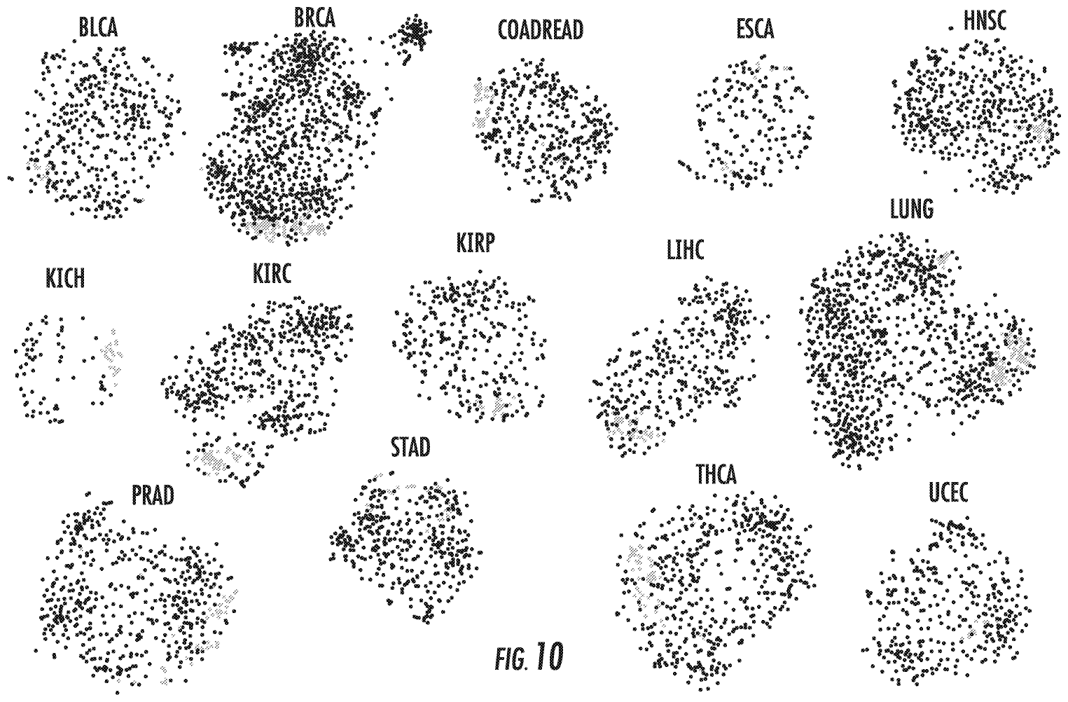

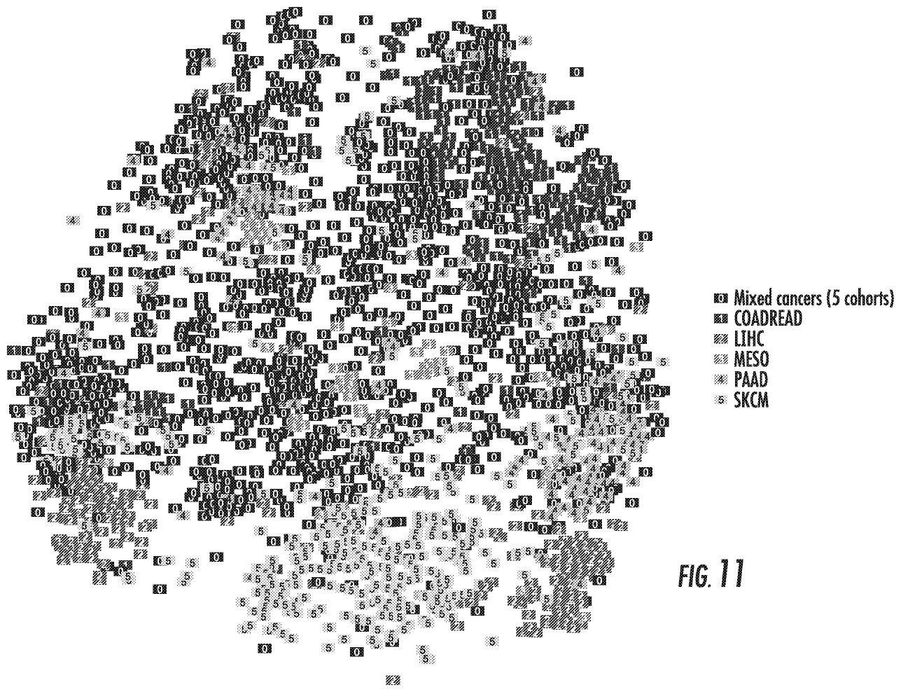

RNA-seq expression data for 9844 tumors (30 cancer types) and 716 matched normal tissues were obtained from The Cancer Genome Atlas (TCGA). Relative expression of RPTs was calculated for all samples and first analyzed using PCA. Normal tissue samples could, to a modest degree, be distinguished by their RPT expression patterns, though many tissue types demonstrated considerable overlap (FIG. 3A and FIG. 9A). Patterns of RPT expression in tumors were even more heterogeneous, and most cancer cohorts did not cluster discretely (FIG. 3B).

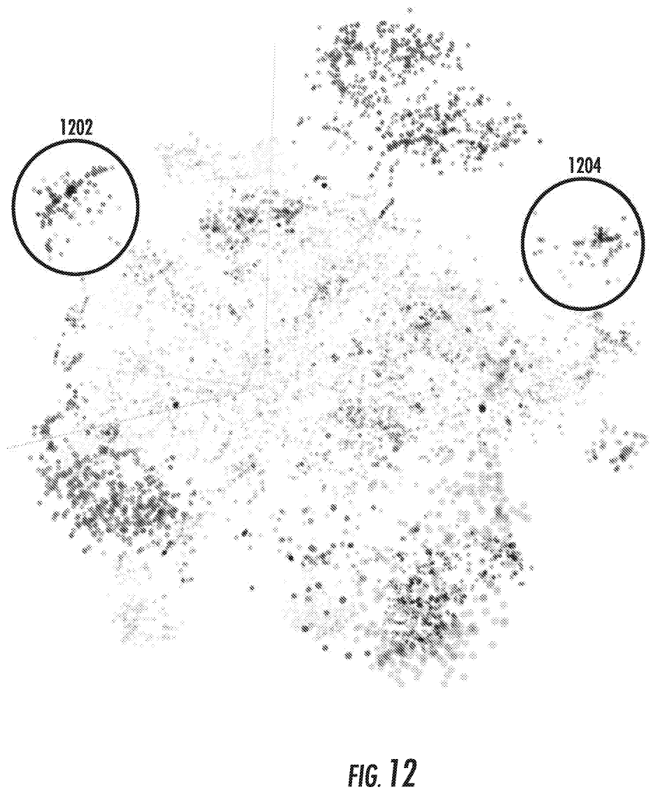

Samples were then analyzed with t-SNE, which more clearly identified clusters of variation due to its ability to identify non-linear relationships between RPTs (FIG. 3A and FIG. 3B) and FIG. 9B). Clustering of normal tissue samples correlated near perfectly with tissue type. Tumors also demonstrated clustering that strongly associated with tissue type, with 20 cohorts possessing largely distinct, non-overlapping clusters of tumors. When both normal tissues and tumors were analyzed together with t-SNE, samples also generally grouped into large clusters according to tissue type. Normal tissues, however, localized into smaller sub-clusters distinct from tumors (FIG. 3C and FIG. 10). Thus, while samples nearly always possessed RPT expression specific to their tissue type, normal tissues and tumors could be readily distinguished from one another.

Five cohorts—cholangiocarcinoma (CHOL), lung (LUNG), bladder (BLCA), cervical (CESC), and uterine carcinosarcoma (UCS)—were comprised of tumors that lacked tissue-specific RPT expression profiles and did not form distinct clusters. These tumors displayed significant overlap with each other as well as with tumors from the remaining five cohorts—liver (LIHC), colorectal (COADREAD), mesothelioma (MESO), pancreatic (PAAD), and skin cutaneous melanoma (SKCM)-which otherwise clustered distinctly from one another (FIG. 11). Additionally, two clusters of tumors were found that did not associate with tissue of origin (see FIG. 12, groups 1202 and 1204). The first —1202—contained 143 tumors from 15 cohorts, 98% of which had amplification and relative up-regulation of RPL19, RPL23, and ERBB2 (Her2/Neu). The second—1204—contained 77 tumors from 12 cohorts with no discernable or unifying RPT expression pattern.

t-SNE Identifies Sub-Types of RPT Expression within Cancer Types

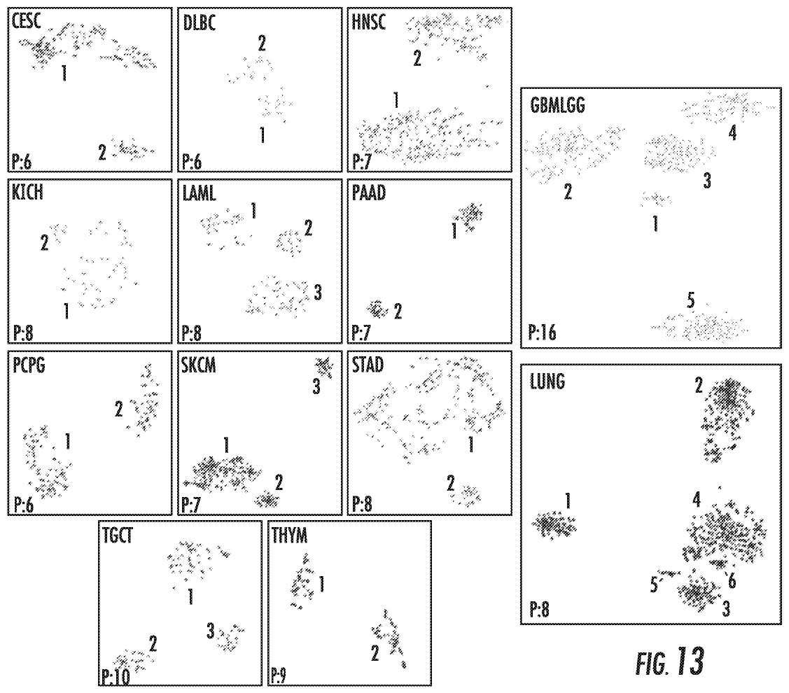

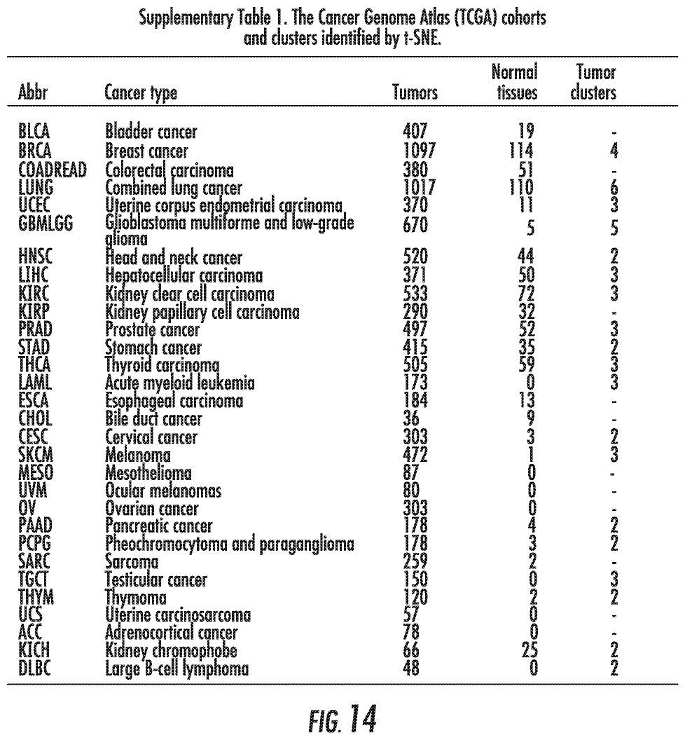

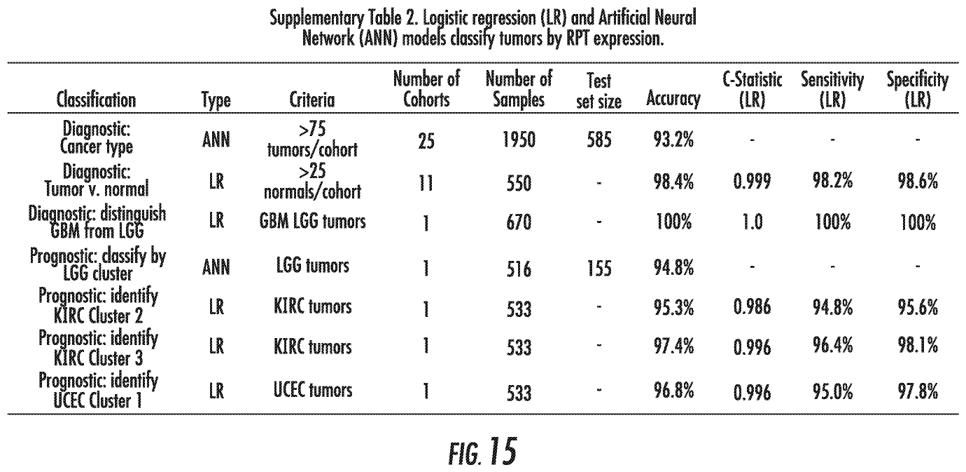

Analyzed individually, 19 of 30 cancer types demonstrated sub-clustering of RPT expression with t-SNE (FIG. 3D), FIG. 13 and Supplementary Table 1 in FIG. 14). Graphing RPT relative expression by cluster using a 3D area map illustrated the different patterns of expression detected by t-SNE (FIG. 3E). In some cases, these clusters differed from one another in the expression pattern of numerous RPTs, as with Clusters 1 and 3 of prostate cancer. In other cases, expression patterns appeared to be dominated by the differential relative expression of one or two RPTs, as with prostate cancer Cluster 2 and HCC Cluster 3, both of which possess tumors that overexpress RPL8 and under-express RPL3 (FIG. 3E). While all clusters were distinct from normal tissues (FIG. 3C and FIG. 10), some clusters were more similar to normal tissues than others, such as prostate cancer Cluster 1 and HCC Cluster 1 (FIG. 3E).

Classification Models

While t-SNE analyses are useful for visualization and pattern discovery, they do not alone provide a direct means for classification of future samples. Thus, with the knowledge that RPTs have both tissue- and tumor-specific expression patterns, various tumor classifier models were constructed based on these patterns. The constructed models consisted of both artificial neural network (ANN) and logistic regression (LR) classifiers, and are listed in Supplementary Table 2 in FIG. 15. An ANN model classified tumors by RPT content according to their tissue of origin on a separate test set with 93% accuracy. Similarly, a LR model distinguished tumors from normal tissues with >98% accuracy. Other LR models could distinguish glioblastoma multiforme tumors from other brain cancers with 100% accuracy and stratify both uterine and kidney clear cell tumors according to prognostic group with >95% accuracy.

Characterizing Tumor Clusters Identified by t-SNE

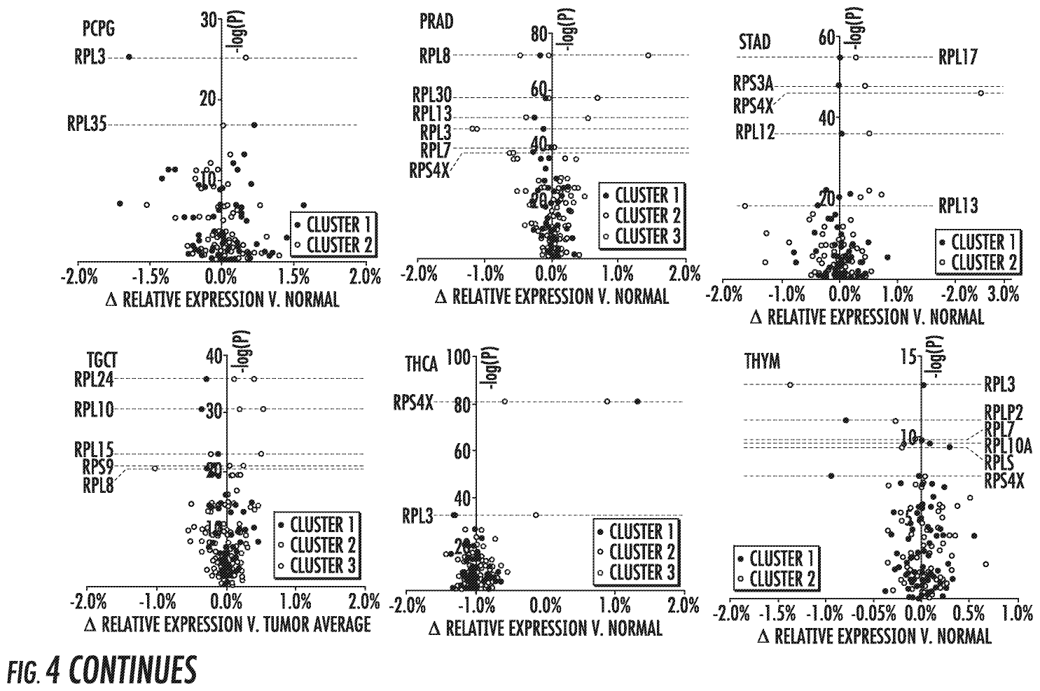

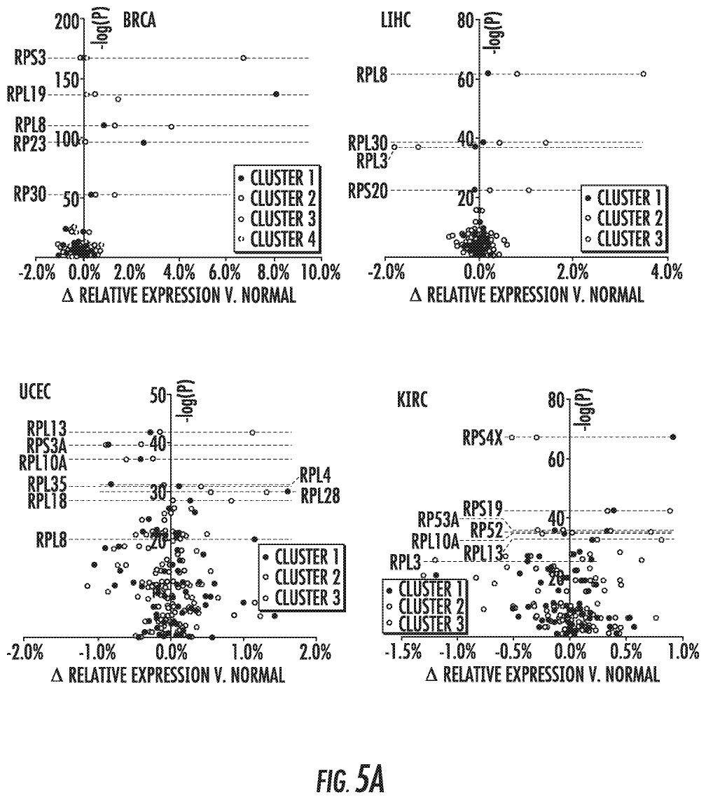

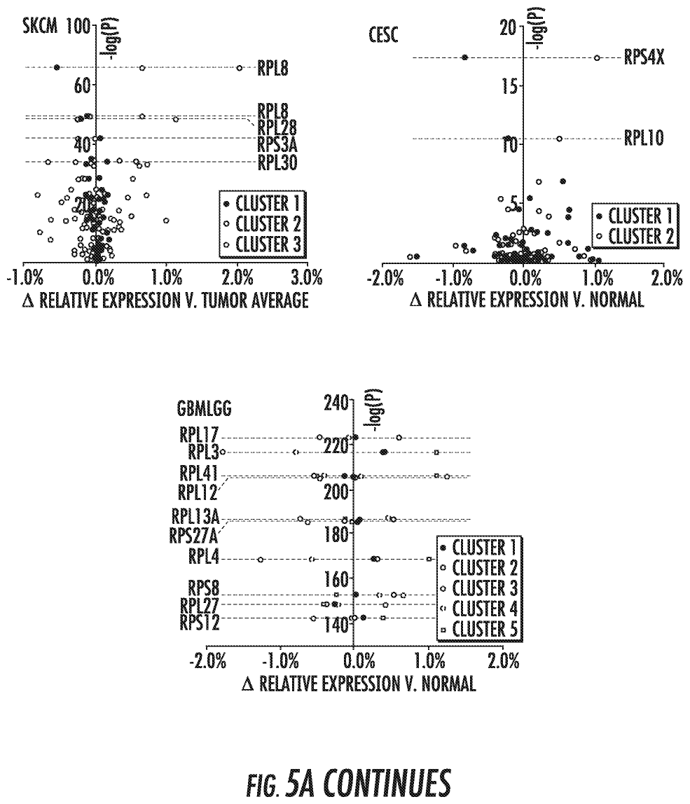

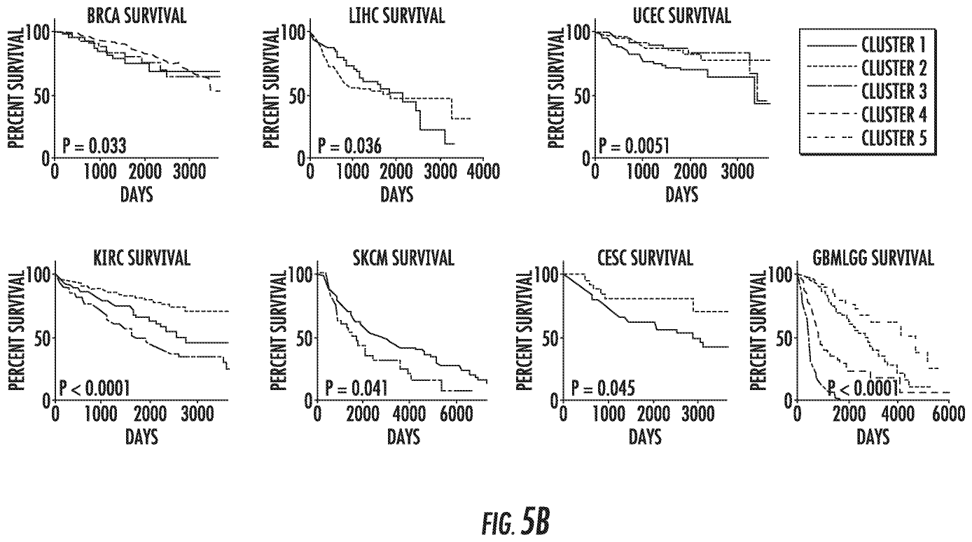

In order to quantify the differences in RPT expression that exist between clusters of tumors identified by t-SNE, RPT relative expression was compared between clusters of tumors with Analysis of Variance (ANOVA) and graphed with volcano plots (FIG. 4 and FIG. 5A). Small but highly significant differences in the expression of dozens of RPTs occurred in nearly every tumor cluster (P as low as 10⁻²²⁰). As was the case with prostate cancer and HCC, expression patterns in clusters were often dominated by particularly significant differences in expression of one or two RPTs, most commonly RPL3, RPS4X, RPL8, RPL30, and RPL13. Other tumor clusters, notably those involving the uterus, brain, and lung, possessed more complex differences involving many RPTs (FIG. 4 and FIG. 5A).

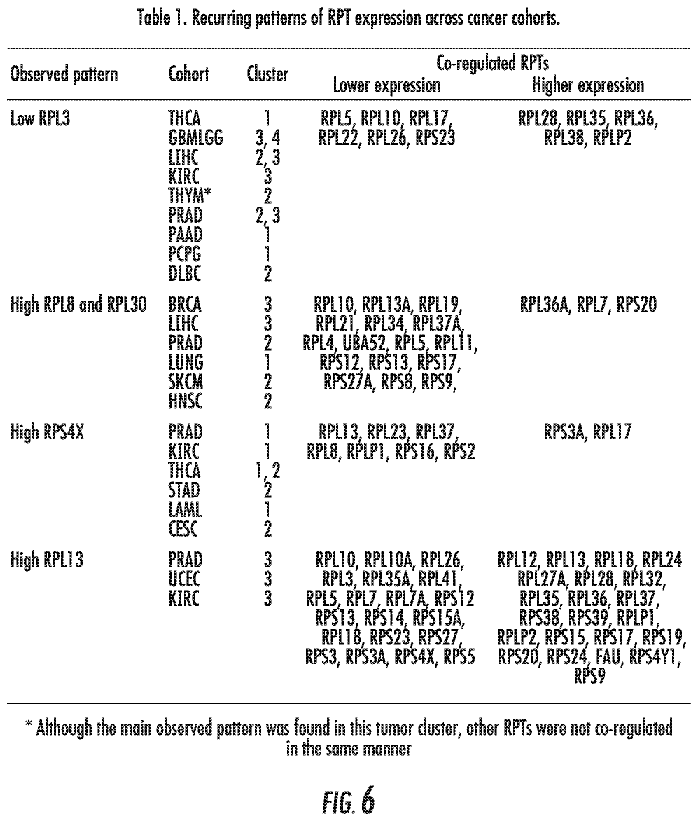

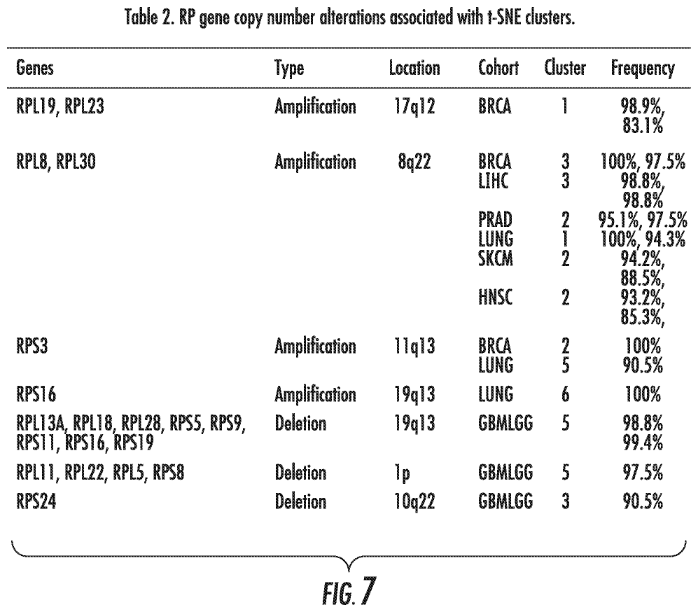

Several recurrent alterations in RPT expression were found among the 19 cancer cohorts with sub-clustering (Table 1 in FIG. 6). Nine of these clusters, arising from thyroid, brain, liver, kidney clear cell, thymoma, prostate, pancreatic, pheochromocytoma and paraganglioma, and B-cell lymphoma, contained tumors with low relative expression of RPL3. These clusters also shared expression patterns with other RPTs, including the relative down-regulation of RPL5 and up-regulation of RPL36 and RPL38. Excluding thymomas, all other tumor clusters with low RPL3 also shared 11 other similarly co-regulated RPTs. Additionally, six cancer cohorts—prostate, breast, liver, lung, melanoma, and head and neck—contained tumor clusters distinguished by overexpression of RPL8, RPL30 and RPS20, with shared expression patterns of 19 other RPTs. Relative up-regulation of RPS4X occurred in tumors from six cohorts, all of which showed similar co-expression patterns of nine other RPTs. Finally, tumor clusters overexpressing RPL13 were found in prostate, uterine and kidney clear cell carcinoma and shared similar patterns of expression of 42 other RPTs (FIG. 4 and FIG. 5A) and Table 1 in FIG. 6).

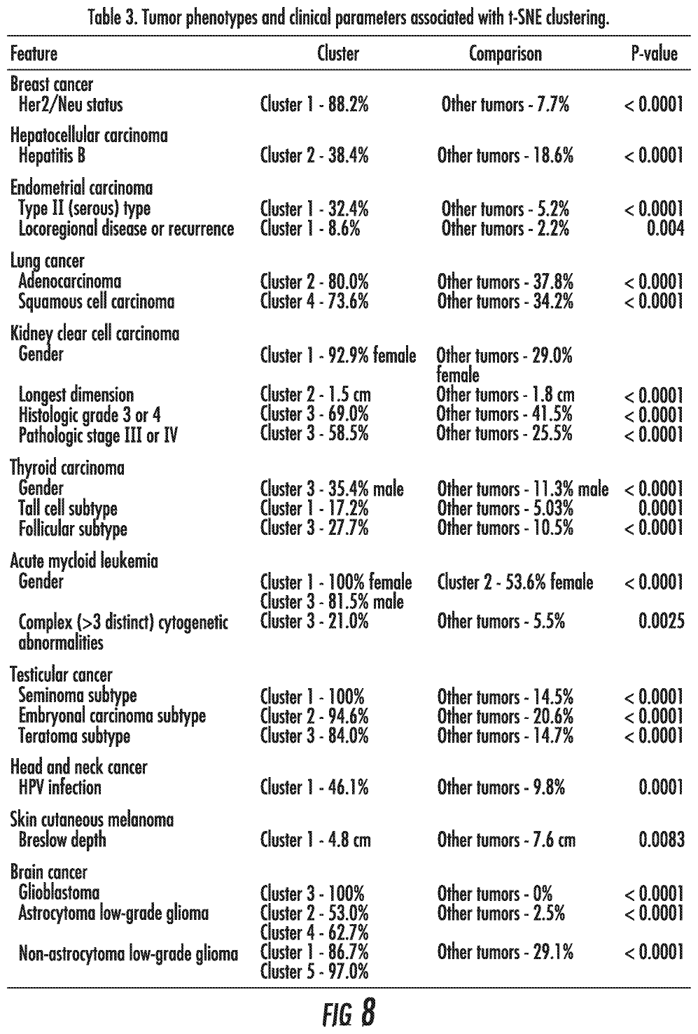

In some cases, RP gene copy number variations (CNVs) were associated with clustering (Table 2 in FIG. 7). Notably, the aforementioned RPL8/RPL30 overexpression pattern strongly correlated with co-amplification of a region on 8q22-24 containing RPL8, RPL30, and MYC. Similarly, an amplicon containing RPL19, RPL23, and ERBB2 (Her2/Neu) was amplified in 99% of the breast cancers in Cluster 1 (Her2/Neu+ tumors). Some tumor clusters associated with specific CNVs to a lesser degree. For example, 48% of tumors in kidney clear cell carcinoma Cluster 3 possessed deletions of RPL12, RPL35, and RPL7A on 9q33-34. Similarly, half of brain cancers in Cluster 1 possessed a 1p/19q13 co-deletion, compared to nearly 100% of tumors in Cluster 5 with this deletion (Table 2 in FIG. 7). Other tumor clusters in various cancer cohorts had differences in overall CNV frequencies. In testicular cancer, 39 RP genes were amplified at different frequencies among the three clusters. Endometrial cancer Cluster 1 and HCC Cluster 2 had more CNVs overall, but no RP gene was amplified or deleted with a frequency of greater than 65% in any given tumor cluster.

Many tumor clusters—each representing a distinct RPT expression pattern —significantly associated with various clinical parameters, molecular markers, and tumor phenotypes (Table 3 in FIG. 8). This was particularly true for brain cancer, testicular cancer, thyroid cancer, lung cancer, and endometrial cancer. Tumor clusters in HCC and head and neck cancers strongly correlated with etiologically-linked infections. For example, chronic hepatitis B infection was 2-fold more common in HCC patients with Cluster 2 tumors compared to other HCC patients. Similarly, chronic HPV infection was 4.7-fold more frequent in head and neck cancer patients with Cluster 1 tumors compared to other patients in this cohort. Patient gender also associated with tumor clustering to varying but significant degrees in kidney clear cell carcinoma and AML. Notably, these clusters also associated with differential relative expression of the X-chromosome encoded RPS4X. Other clinical markers and tumor phenotypes significantly associated with tumor clustering can be found in Table 3 in FIG. 8.

Tumor clusters were often predictive of survival, including some clusters that did not significantly associate with any other known tumor subtype (FIG. 5B). For example, Clusters 2 and 4 of the brain cancer cohort, which could not otherwise be distinguished by any known clinical parameter or tumor subtype, possessed vastly different survival patterns. Other cancer cohorts with significant survival differences among clusters included breast, liver, endometrial, kidney clear cell, melanoma, and cervical cancers.

Discussion

By investigating expression patterns of individual RPTs and utilizing more traditional and less powerful linear forms of dimensionality reduction such as PCA, previous studies have found modest evidence of tissue-specific patterning of RPT expression in some normal tissues³. Extending these types of analyses to tumors has been largely unfruitful, presumably due to the complex regulation of RPT expression and because many of the RPT relationships are non-linear. As shown here, however, the machine learning algorithm t-SNE provides a more elegant and robust dimensionality reduction that better highlights distinct patterns of RPT expression in both tumors and the normal tissues from which they arise.

Consistent with more restricted and tentative conclusions of previous findings, the results using t-SNE demonstrate that RPT expression patterns are not only tissue-specific but provide the ability to define tissue and tumor differences with a heretofore unachievable degree of resolution and confidence. The small cluster of 77 neoplasms that did not associate with their respective tissue clusters (FIG. 12) may represent either a subset of tumors that have lost control of their underlying tissue-specific expression patterns or that originated from a minority subpopulation of normal cells whose RPT expression is not representative of the remainder of the tissue.

In addition to their tissue-specific patterning, virtually all tumors showed perturbations of RPT expression that readily allowed them to be distinguished from normal tissues. In some cancers, the tumor-specific patterning of RPT expression was relatively homogeneous and could not otherwise be subcategorized. Most cohorts, however, were comprised of subgroups of tumors with distinct RPT expression patterns, all of which remained distinguishable from normal tissue. The fact that many of these patterns correlated with molecular and clinical features implicates RPT expression patterns in tumor biology.

Aside from potentially altering translation, the notion that altered RP expression might influence the behaviors of both normal tissues and tumors is not new. In the ribosomopathies, the binding of any one of about a dozen RPs to MDM2 with subsequent stabilization of p53 is thought to underlie bone marrow failure^{6,9,10}. It has been proposed that subsequent circumvention of this p53-mediated senescence by mutation and/or dysregulation of the p19^{ARF}/MDM2/p53 pathway is responsible for the propensity for eventual neoplastic progression³⁰. In cancers, the binding of free RPs to MDM2 has been shown to mediate the response to ribosomal-stress-inducing chemotherapeutics such as actinomycin D and 5-fluorouracil^{19,31,32}.

Individual RPs have also been associated with specific tumor phenotypes. For example, RPL3 regulates chemotherapy response in certain lung and colon cancers, associates with the high-risk neuroblastoma subtype, and may have a role in the acquisition of lung cancer multidrug resistance^{18,20}. Breast cancers with elevated expression of RPL19 are more sensitive to apoptosis mediated drugs that induce endoplasmic reticulum stress¹². RPS11 and RPS20 have been proposed as prognostic markers in glioblastoma¹⁵ and the down-regulation of RPL10 correlates with altered treatment response to dimethylaminoparthenolide (DMAPT) in pancreatic cancer²¹.

The results also extend the findings of previous studies by demonstrating that in the vast majority of cancers, subsets of RPTs are expressed coordinately and have additional interpretive power when examined in the context of global RPT expression patterning. This suggests that further insights into the roles RPTs have in tumor development may be revealed by evaluating RPT relative expression. For example, the regulation of chemotherapy response by RPL3 may be found to occur in other cancer types once the expression of RPL3 relative to other RPTs has been taken into account. The apparent crucial role of RPT patterning in tumors may explain why a previous study found conflicting results when examining the expression of individual RPs in tumors¹³.

The results suggest a more ubiquitous role for RPL3 in regulating tumor phenotypes, beyond that already described in colorectal carcinoma, lung cancers, and neuroblastoma¹⁸⁻²⁰. Of the recurring RPT expression patterns discovered by t-SNE, the pattern associated with RPL3 down-regulation occurred most frequently, involving tumors from nine cancer cohorts. Many clusters of tumors with down-regulated RPL3 possessed inferior survival, including those from liver, kidney clear cell, and brain cancers. The fact that relative down-regulation of RPL3 occurred in these tumor clusters with predictable expression of 11 other RPTs suggests that RPL3 may be acting in concert with these other identified RPs to exert its effects.

Other recurring patterns of RPT expression across cancer cohorts involved RPS4X, RPL13, RPL8 and RPL30 (Table 1 in FIG. 6). Altered RPS4X expression, found in six cancer cohorts, associated with unique expression of nine other RPTs, strongly suggesting an underlying coordinated expression. As with RPL3, deregulated RPS4X has been previously associated with various tumors and tumor phenotypes, including subgroups of colorectal carcinoma, a myelodysplasia risk signature and poor prognosis in bladder cancer^{14,17,33}. Interestingly, some of the tumor clusters with altered RPS4X expression were comprised of a greater proportion of females than males (Table 1 in FIG. 6 and Table 3 in FIG. 8), perhaps reflecting the fact that the RPS4X gene resides on chromosome X. Although the cause of perturbed RPS4X expression in these tumor clusters is unknown, altered methylation patterns on chromosome X have been described in different subsets of cancers^{34,35} and could be responsible for the RPS4X expression patterns detected by t-SNE.

Unlike RPL3 and RPS4X, the role of RPL13 in tumor development is less clear. Activation of RPL13 has been described in a subset of gastrointestinal malignancies and correlated with greater proliferative capacity and attenuated chemoresistance³⁶, but further evidence for a role of RPL13 in tumor development is lacking. Furthermore, clinical correlations of the prostate, uterine and kidney cancer t-SNE clusters described here with relative overexpression of RPL13 were inconsistent. Uterine cancers with high relative RPL13 tended to correlate with favorable survival, whereas prostate cancers with high RPL13 showed no differences in prognosis or clinical features, and kidney clear cell carcinomas with high RPL13 tended to be of higher pathologic grade and conferred significantly poorer survival (Table 1 in FIG. 6, Table 3 in FIG. 8, and FIG. 5B). The fact that these clusters shared similar patterning of 42 other RPTs, however, suggests that the inciting factors responsible for higher RPL13 expression are not only shared by these tumors but coordinately regulate a common subset of RPTs.

In some cases, RPT expression patterns could be accounted for in part by CNVs, as exemplified by the recurrent RPL8 and RPL30 overexpression pattern (Table 1 in FIG. 6 and Table 2 in FIG. 7). Virtually all tumors with this expression pattern possessed co-amplification of a region on 8q22-24 that includes RPL8, RPL30 and the oncogene MYC. Amplification of this region has been previously described in breast cancers and correlates with chemoresistance and metastasis³⁷⁻³⁹. The results indicate that this amplification and the ensuing overexpression of RPL8 and RPL30 also occurs in subsets of melanoma, liver, prostate, lung, and head and neck cancers. CNVs in RPL19 and RPL23 in breast cancer (Table 2 in FIG. 7) likely occur due to their co-amplification with ERBB2 on 17q12. Over expression of RPL19 has previously been described in a subset of breast cancers¹². The small cluster of 144 tumors that did not group according to tissue of origin (FIG. 12), comprised of tumors from 15 cohorts, also shared amplification of this region on 17q12, indicating that this CNV is not restricted to breast cancers and ultimately affects global RPT expression patterning. Amplification of a region on 11q13 that contains RPS3, occurring in a cluster of breast cancers and HCCs, has been previously described in both cancers and is thought to confer unfavorable prognosis due to amplification of the oncogene EMS1 in this region^{40,41}. The co-deletion of 19q13 with regions of 1p, which include numerous RP genes, has been described in low-grade gliomas and correlates with a favorable prognosis^{42,43}.

The co-overexpression RPS25 and RPS4X detected in one cluster of AML (FIG. 4) has been previously identified as contributing to the poor risk signature in myelodysplastic syndrome³³. This also associated with significant differential expression of 37 RPTs, which is consistent with the finding that RPS25 and RPS4X overexpression occur within the context of a larger and coordinated pattern of RPT expression. The RPS25 and RPS4X overexpressing AML cases likely possess a similar molecular alteration to those with the poor risk signature in MDS.

Collectively, the findings provide strong evidence to support the notion that RPT regulation by both tumors and normal tissues is complex, ordered, and highly coordinated. Although the means by which altered RPT patterns influence the pathogenesis and/or behavior of tumors remain incompletely understood, several non-mutually exclusive mechanisms can be envisioned. First, changes in RP levels may influence overall ribosome composition, affecting the affinity for certain classes of transcripts and/or the efficiency with which they are translated. One such class of transcripts may be those with IRES elements, cis-regulatory sequences found in the 5′-untranslated regions of more than 10% of cellular mRNAs. IRES elements are found with particularly high frequency on transcripts encoding proteins involved in cell cycle control and various stress responses. Efficient translation of these IRES-containing transcripts has been shown to depend on the presence of specific RPs, notably RPS25, RPS19 and RPL11⁴⁴⁻⁴⁶. Changes in ribosome affinity for IRES elements have been shown to reduce translation of tumor suppressors such as p27 and p53 and to promote cancer development⁴⁷.

RPs may also influence cancer development via extra-ribosomal pathways. In addition to their promotion of p53 stability mediated by binding to and inactivating MDM2, specific RPs have been shown to inactivate Myc; to inhibit the Myc target Lin28B; to activate NF-κB, cyclins, and cyclin-dependent kinases and to regulate a variety of other tumorigenic functions and immunogenic pathways^{4,5}.

In addition to providing evidence that tumors may use RPs to direct tumor phenotypes, the findings leverage the tissue- and tumor-specificity of RPT expression to generate highly sensitive and specific models that allow for precise tumor identification and sub-classification (Supplementary Table 2 in FIG. 15). Clinically, these might be useful for determining the tissue of origin of undifferentiated tumors and for predicting long-term behaviors in otherwise homogeneous cancers such as in kidney clear cell carcinoma and those of the central nervous system (FIG. 5B). With more samples and further refinement to ANN structures, future iterations of these models will likely have even greater discriminatory power.

A limitation of using data from TCGA is the fact that transcript expression does not always correlate with protein expression, particularly in cancers⁴⁸⁻⁵⁰. Thus, it is difficult to predict how the different tissue-specific RPT expression patterns identified correlate with actual protein expression in these cancers and/or with the numerous post-translational modifications that can alter RP behaviors. As this is a cross-sectional study, it is also recognized that causality cannot be inferred and it remains unknown whether altered RPT expression is an early or late event in tumorigenesis despite its predictive value. Further molecular analyses of the identified t-SNE clusters with whole-transcriptome sequencing data, pathway analysis, whole-genome DNA mutation data, and DNA methylation patterning may offer additional insights into the biological mechanisms that link altered RPT expression with tumor phenotypes.

In summary, machine learning-based approaches have been used to determine that RPTs are expressed with distinct patterning across tissue types. This tissue-specificity persists in tumors, yet normal tissues and tumors can be readily distinguished from one another with high accuracy and confidence. Many cancers can be further sub-categorized into heretofore unrecognized, yet clinically important, subtypes based only upon RPT expression patterns. Several patterns of RPT expression recur across cancer types, suggesting common underlying and regulated modes of transcriptional regulation. The results indicate that the expression of RPTs in tumors is biologically coordinated, clinically meaningful, and can be leveraged to create potential clinical tools for tumor classification and therapeutic stratification.

Materials and Methods

Accessing Ribosomal Protein Transcript Expression Data

RNA-seq whole-transcriptome expression data for 9844 tumors and 716 normal tissues from The Cancer Genome Atlas (TCGA) was accessed using the UCSC Xenabrowser. Only primary tumors were included for analysis, apart from the melanoma (SKCM) cohort, as the vast majority of tumors with sequencing data in this cohort were metastatic (78%). For each of the 30 cancer cohorts, RNA-seq data was selected according to the label “gene expression RNAseq (polyA+ IlluminaHiSeq).” “IlluminaGA” RNA-seq expression data was used for the cohort Uterine Corpus Endometrial Carcinoma (UCEC), as this group of data had more samples than the “IlluminaHiSeq” group. For all cancer cohorts, expression data for 80 cytoplasmic RP genes were extracted and base-two exponentiated, as the raw RPKM (Reads Per Kilobase per Million mapped reads) expression data was stored log-transformed. The sum of total RPKM counts for all ribosomal protein genes were calculated for each sample, and relative expression of each RP gene in a sample was calculated by dividing the RPKM gene expression by this summed expression.

Visualizing Ribosomal Protein Transcript Expression

Principal component analyses and t-SNE analyses of RPT relative expression in normal tissues and tumor samples were performed using TensorFlow r1.0 and Tensorboard (https://tensorflow.org). TENSORFLOW and TENSORBOARD are open-source software developed by GOOGLE, INC. of Mountain View, Calif. t-SNE analyses were performed at a learning rate (epsilon) of 10 with 5000 iterations or until the visualization stabilized. t-SNE was initially performed in two dimensions for all analyses; data sets that could not be cleanly visualized with two dimensions, particularly those with a large number of samples, were visualized with three-dimensional t-SNE. Multiple analyses were performed with perplexity settings varying between 6-15 for all individual cohort analyses and 10-30 for all grouped cohort analyses, with final perplexity settings for each analysis chosen to maximize cluster distinctions. Clusters of at least 10 samples which distinctly separated visually from other samples were named and samples from these clusters were identified. 3D area maps of RPT relative expression were generated using Microsoft Excel, with each sample listed across the x-axis, RPTs listed across the z-axis, and relative expression of each RPT across the y-axis.

Comparing t-SNE Clusters

Relative expression of RPTs were compared between t-SNE clusters with Analyses of Variance (ANOVA) using R version 3.3.2 (http://www.R-project.org/). ANOVA p-values were log₁₀-transformed and used to generate Volcano plots comparing expression patterns between clusters. Volcano plots were graphed with Graphpad Prism 7 (GraphPad Software, Inc., La Jolla, Calif.).

Clinical and survival data for each TCGA cancer cohort were accessed again using the UCSC Xenabrowser under the data heading “Phenotypes.” For each cohort, survival curves of tumors in each t-SNE cluster were compared with Mantel-Haenszel (log-rank) and Gehan-Breslow-Wilcoxon methods using Graphpad Prism 7. Categorical clinical variables were compared between clusters of tumors with Chi-squared tests. Continuous variables which were normally distributed were compared with t-tests assuming heteroskedasticity, and non-normally-distributed variables were compared with Wilcoxon sign-rank tests. All statistical tests were two-tailed.

Co-Regulated RPTs

Certain groups of RPTs possessed recurring, highly-significant differences between multiple t-SNE clusters, including RPL3, RPL8, RPS4X, and RPL13. For each TCGA cohort with a cluster that possessed significantly different relative expression of one of these transcripts, relative expression of all other RPTs was compared between the identified cluster and other tumors in the same cohort. Co-regulated transcripts were defined as those with consistent differences in relative expression when comparing clusters of interest to other tumors from the same cohort (Table 1 in FIG. 6). For example, five TCGA cohorts had a t-SNE cluster with significant relative overexpression of RPL8 and RPL30. When comparing relative expression of other RPTs between these clusters and other tumors from the same cohorts, all five clusters with high RPL8 and RPL30 also displayed, on average, lower relative expression of RPL10 and higher relative expression of RPL7.

Ribosomal Protein Gene Copy Number Variations (CNVs)

CNV data for TCGA tumors was accessed using the UCSC Xenabrowser under the data heading “copy number (gistic2_thresholded).” Positive values were classified as amplifications, and negative values were classified as deletions. The frequency of amplifications and deletions in RP genes were compared between clusters of tumors in each TCGA cohort using Chi-squared tests and adjusted for 5% false discovery rate. Within each cancer cohort, clusters of tumors with significantly greater incidence of a CNV compared to other tumor clusters, and which possessed >90% incidence of this copy number variation, were included in Table 2 in FIG. 7.

Classification Models

Using RPT relative expression in tumors and normal tissues, classification models were created using both logistic regression (LR) and feed-forward, fully-connected artificial neural networks (ANNs)⁵¹. LR models were used for binary classifiers and developed with Stata SE 14 (StataCorp LP, College Station, Tex.) with c-statistics, sensitivity, and specificity reported in Supplementary Table 2 in FIG. 15. ANN models were generated for classifiers with multiple outcomes (e.g. tissue of origin models) and binary classifiers with a LR model that failed to converge.

ANN models were created and tested using TensorFlow with graphics processing unit (GPU) acceleration on a Titan X Pascal (NVIDIA, Inc., Santa Clara, Calif.). To reduce bias, samples were balanced for both training and testing by cancer cohort such that each training and test set had the same number of samples from each cohort. 60% of data sets were used for training and 10% for validation and hyper-parameter tuning. Hyper-parameter sweeps were used to test all possible combinations of the following: learning rate (0.001, 0.002, 0.005, 0.01), batch size (100, 500, none), dropout rate (0.9, 0.95, 1), hidden layer structure (both one and two layers with sizes varying between 0-200 in increments of 25), and L2 regularization rate (0.00001, 0.0001, 0.001). All ANNs utilized ReLU activation functions. Neural network training performance was monitored with Tensorboard and stopped once validation accuracy had plateaued. The remaining 30% of data comprised a separate test set, which was used to test the final model's classification accuracy once the hyper-parameters were chosen and the model trained. Performance of ANN models on the separate test sets were reported as classification accuracies in Supplementary Table 2 in FIG. 15.

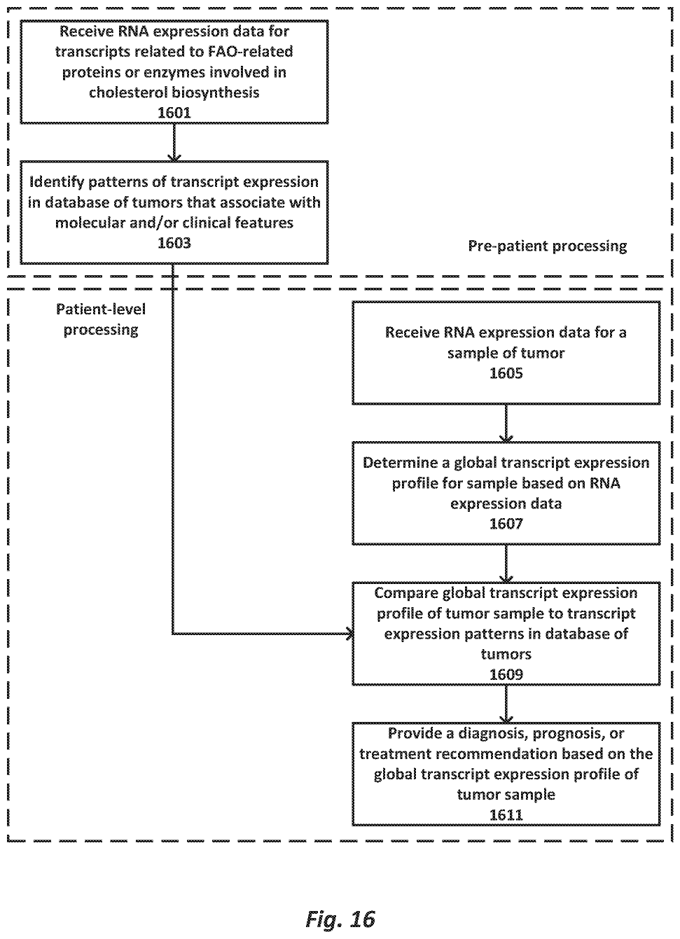

Described herein are methods of bioinformatics. These methods include receiving RNA expression data for a tumor and identifying expression patterns of transcripts based on the RNA expression data. For example, a bioinformatics method is described above with regard to FIG. 1, where expression patterns of ribosomal protein transcripts (RPTs) are identified. This information can be used to identify a tissue of origin and/or provide a diagnosis, prognosis, or treatment recommendation for a patient. As described herein, a machine learning algorithm that is configured to analyze linear and non-linear relationships in a dataset can be used to identify expression patterns of RPTs. Optionally, the machine learning algorithm is t-SNE. This disclosure also contemplates that expression patterns of other transcripts (e.g., transcripts encoding FAO-related proteins or transcripts encoding enzymes involved in cholesterol biosynthesis) can be identified using the bioinformatics methods described herein. The expression patterns of other transcripts can be used to provide a diagnosis, prognosis, or treatment recommendation for a patient. For example, bioinformatics methods are described below with regard to FIG. 16, where expression patterns of cholesterol biosynthesis transcripts or expression patterns of FAO transcripts are identified. This disclosure contemplates that the bioinformatics methods described herein may be used to identify expression patterns in other families of transcripts.

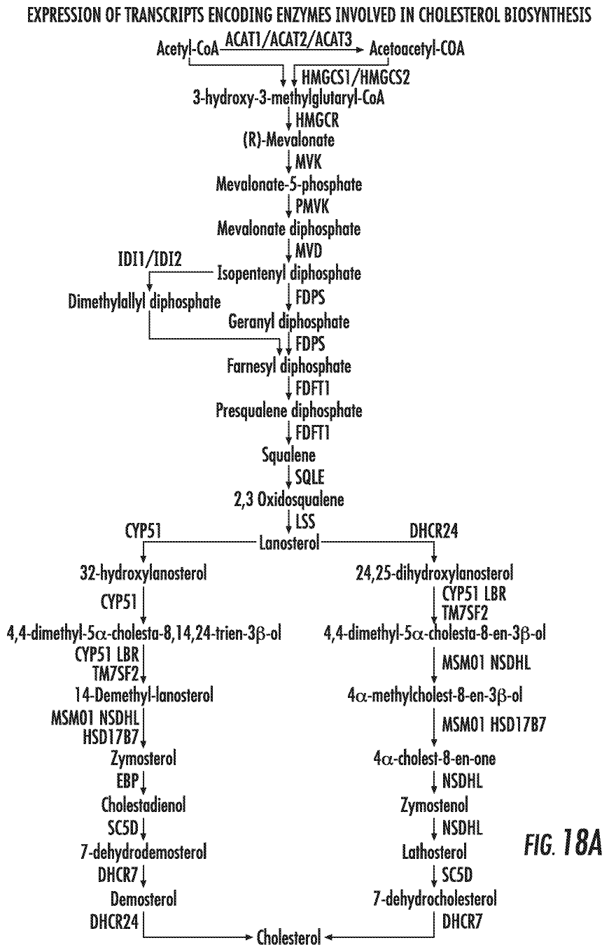

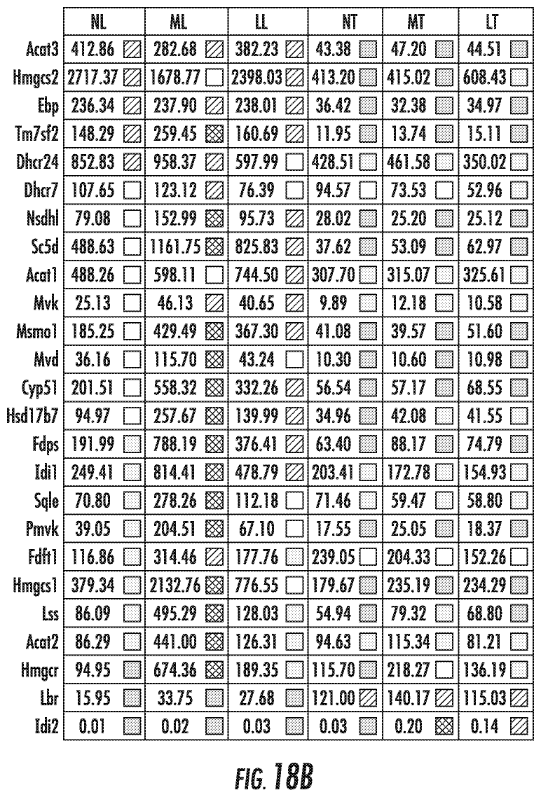

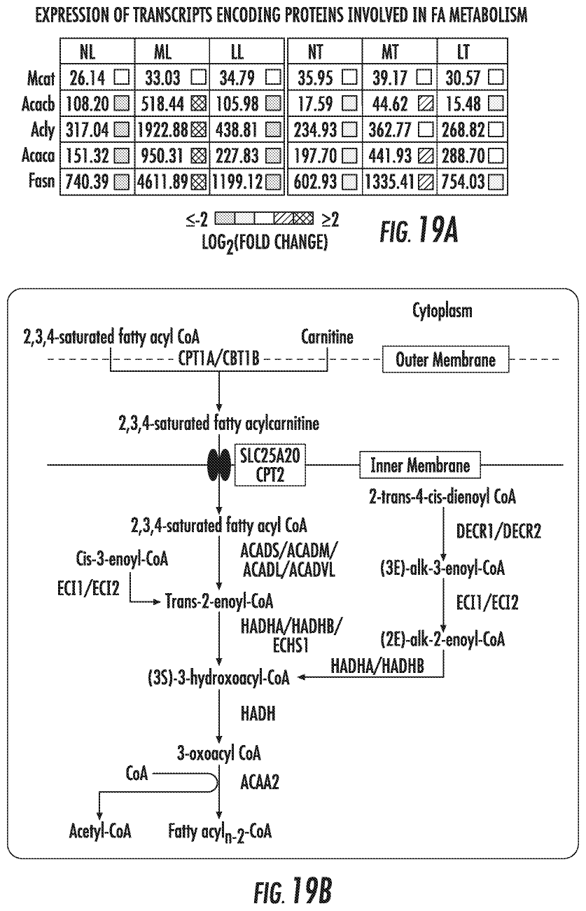

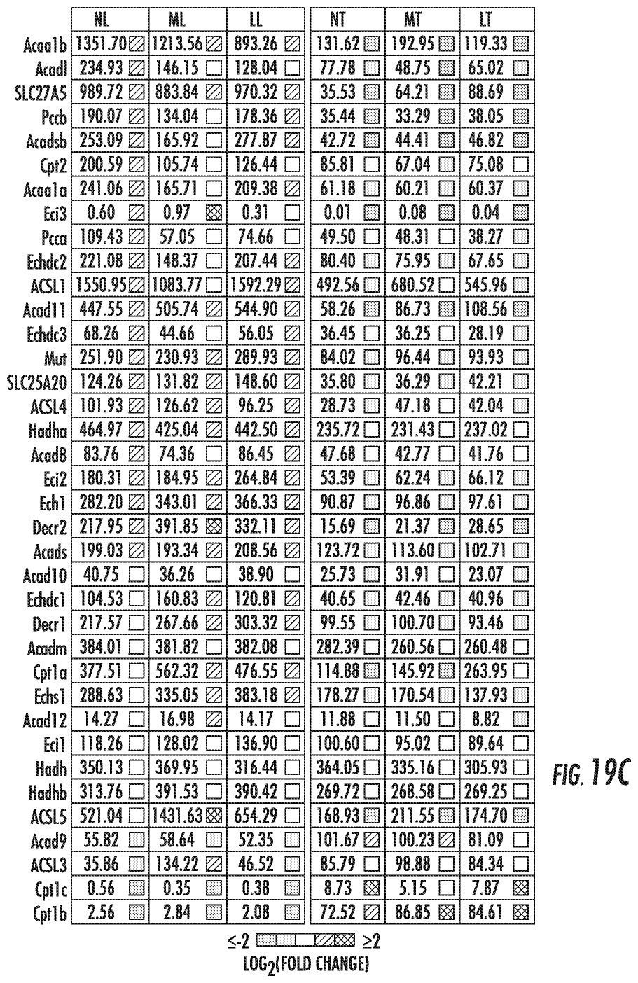

Referring now to FIG. 16, a flow chart illustrating another example operations for a bioinformatics method described herein is shown. FIG. 16 illustrates pre-patient processing steps (e.g., steps 1601 and 1603) and patient-level processing steps (e.g., steps 1605-1611). At 1601, a database of RNA expression data that includes expression of FAO-related proteins or expression of enzymes involved in cholesterol biosynthesis (e.g., RNA-seq, whole transcriptome sequence data, or microarray data) for a plurality of tumors is received or accessed. Optionally, clinical data for the patients from which these tumors derive can also be received or accessed at step 1601. Such a database can include, but is not limited to, The Cancer Genome Atlas (TCGA). At 1605, RNA expression data that includes the expression of FAO-related proteins or expression of enzymes involved in cholesterol biosynthesis for a sample of tumor (sometimes referred to herein as “individual tumor sample”) is received. Example cholesterol biosynthesis transcript expression is shown in FIGS. 18A-18B. Example FAO transcript expression is shown in FIGS. 19A-19C.

In some implementations, the RNA expression data for the individual tumor sample is received, for example, at a computing device (e.g., computing device 200 of FIG. 2). In other implementations, the sample of tumor is optionally received, for example, at a laboratory or other facility for analysis. In this case, the method can include extracting RNA from the sample and isolating FAO-related proteins or enzymes involved in cholesterol biosynthesis from the same. After isolating the proteins and/or enzymes of interest, the RNA expression data can be obtained by sequencing the same. As described herein, techniques for extracting RNA, isolating RNAs, and sequencing are known in the art and are therefore not describe in further detail herein.

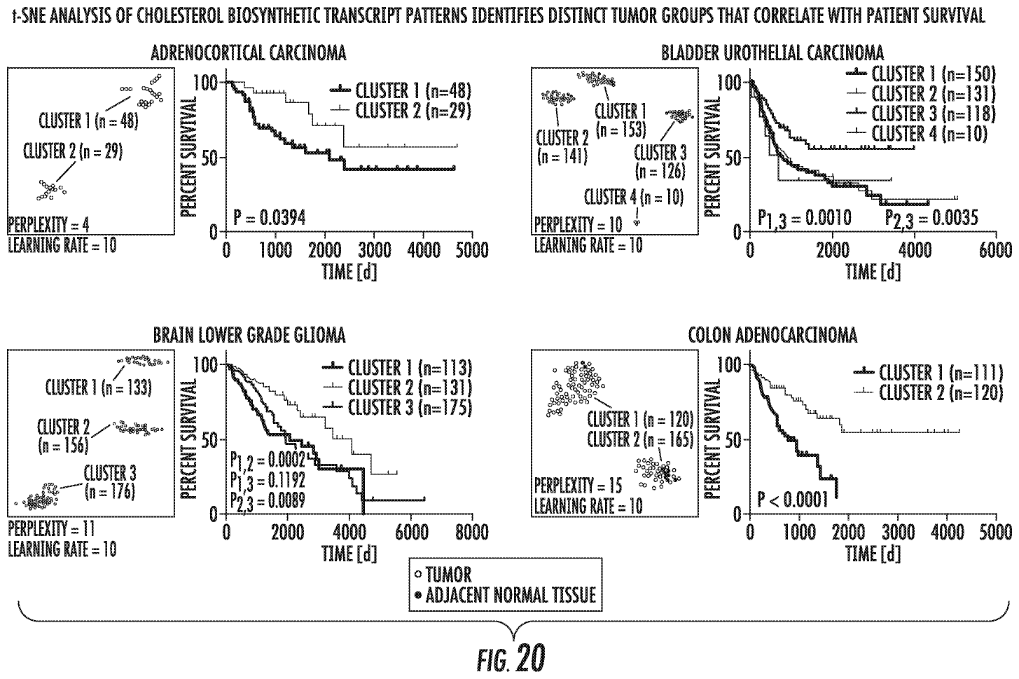

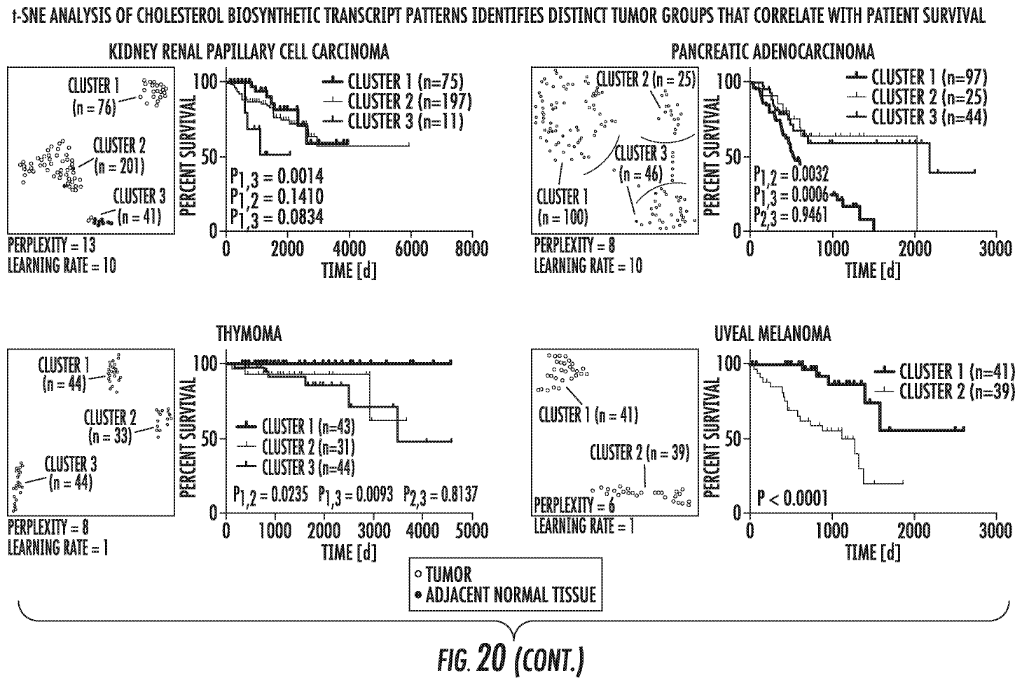

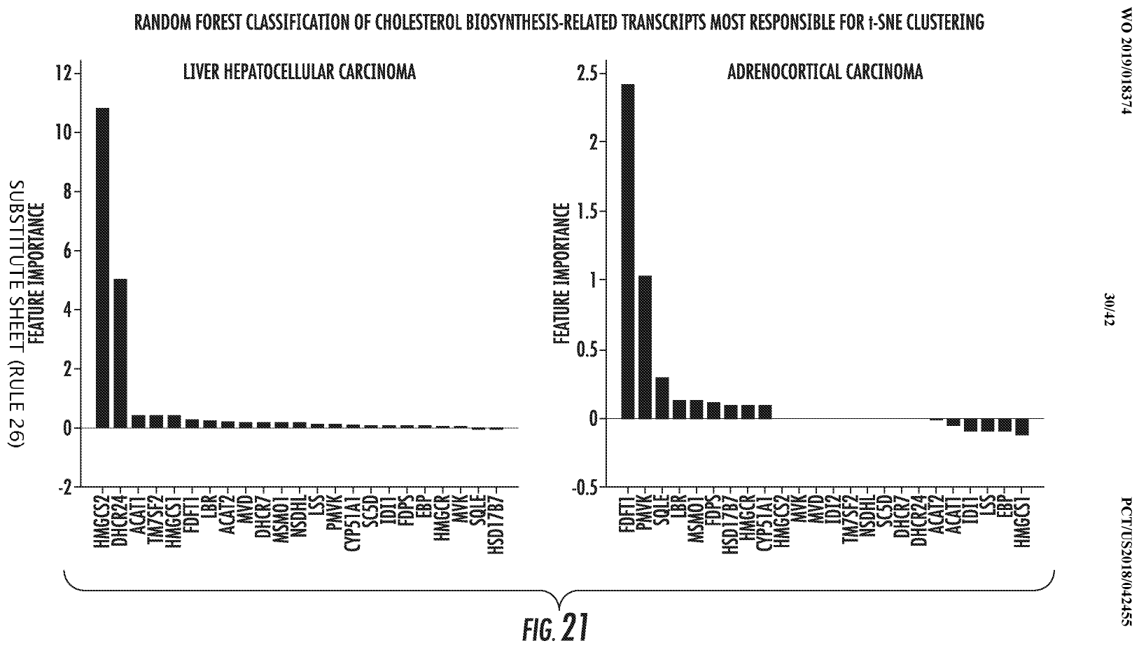

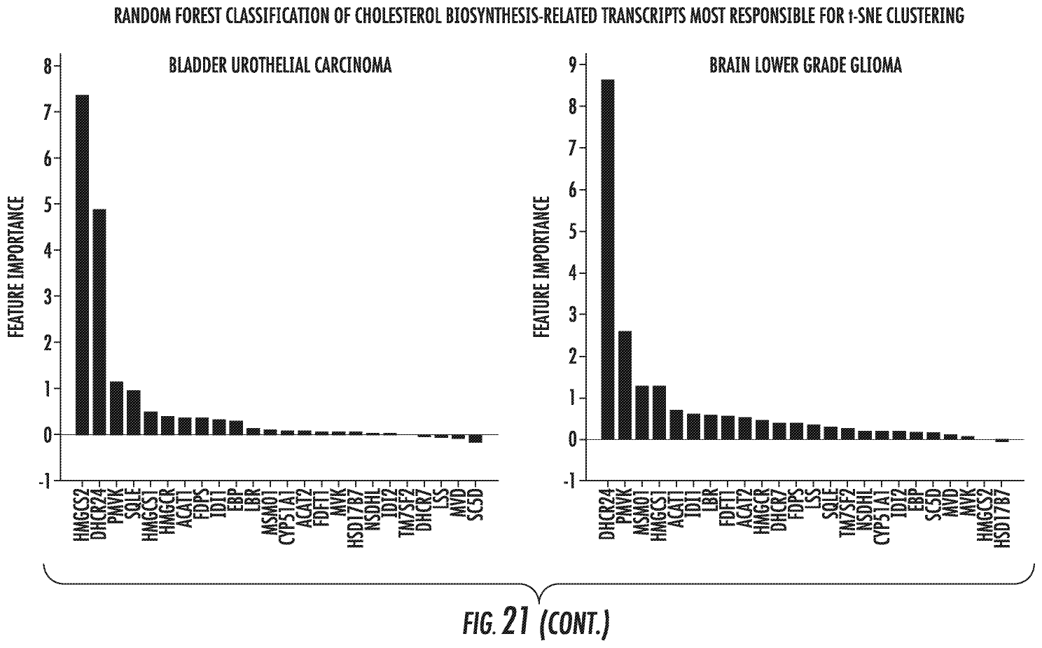

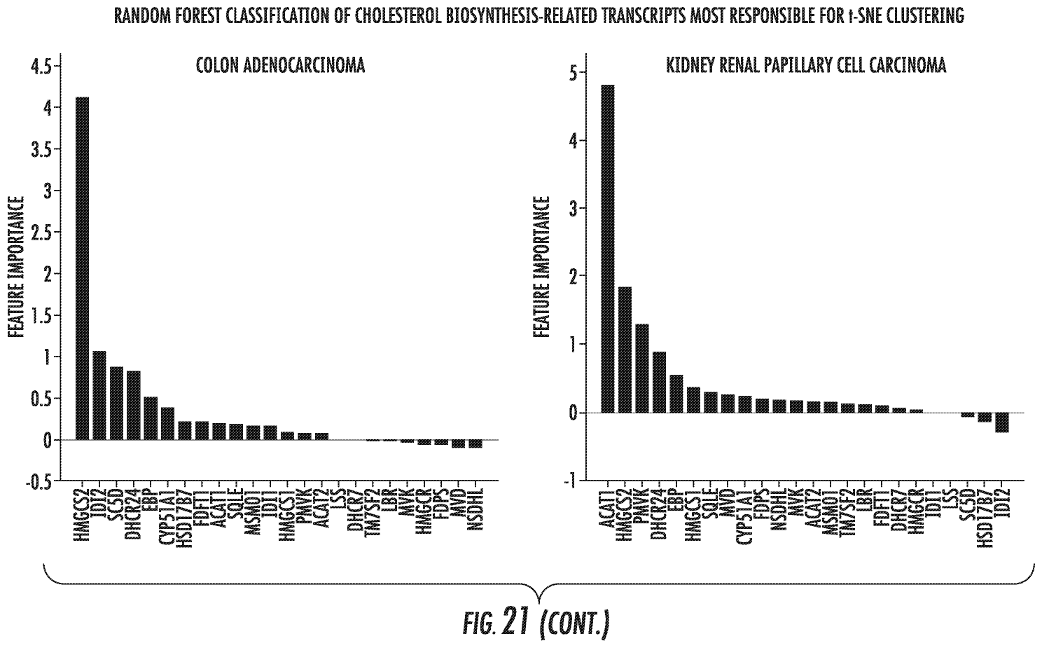

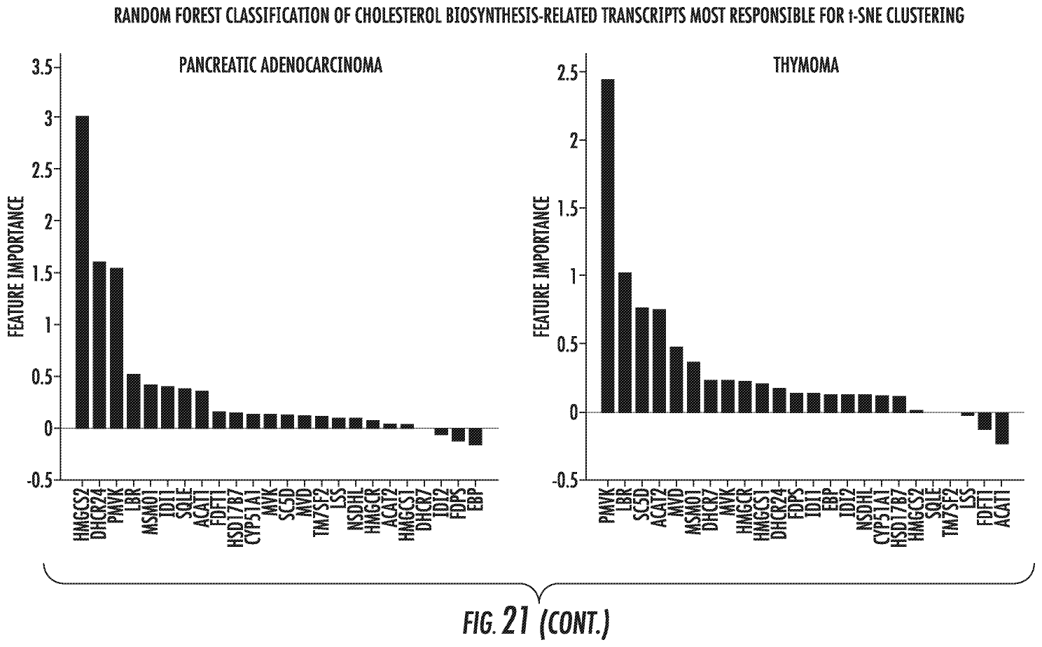

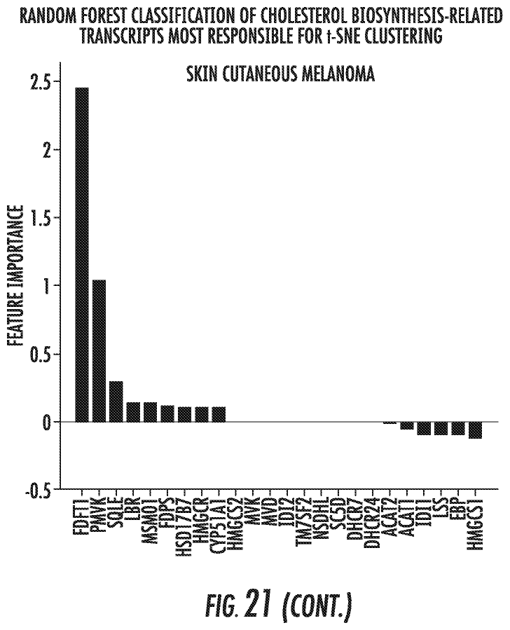

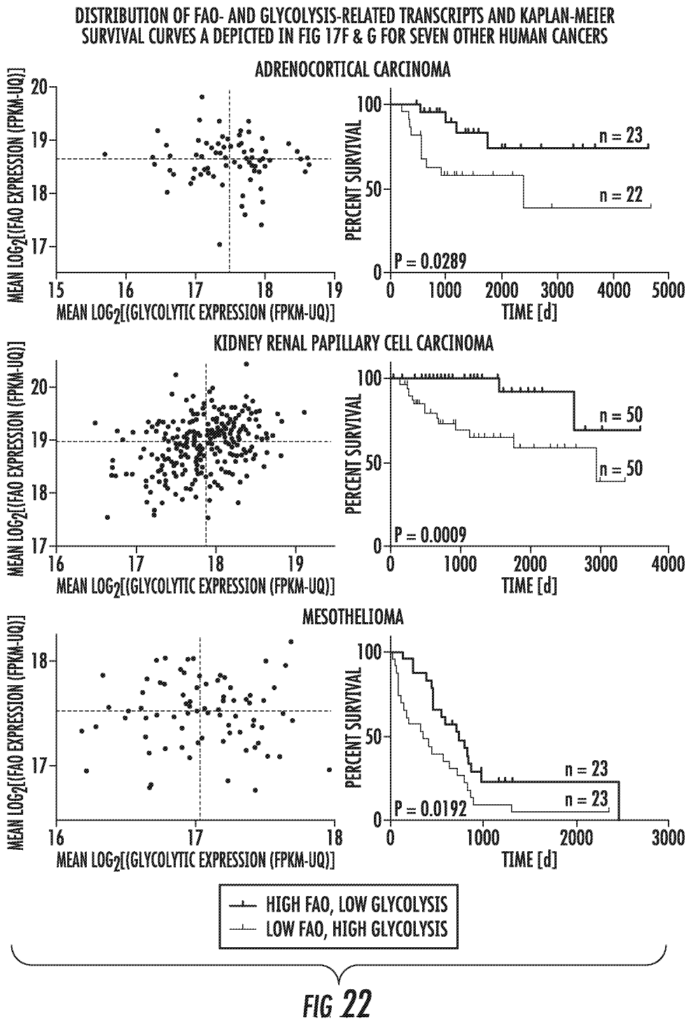

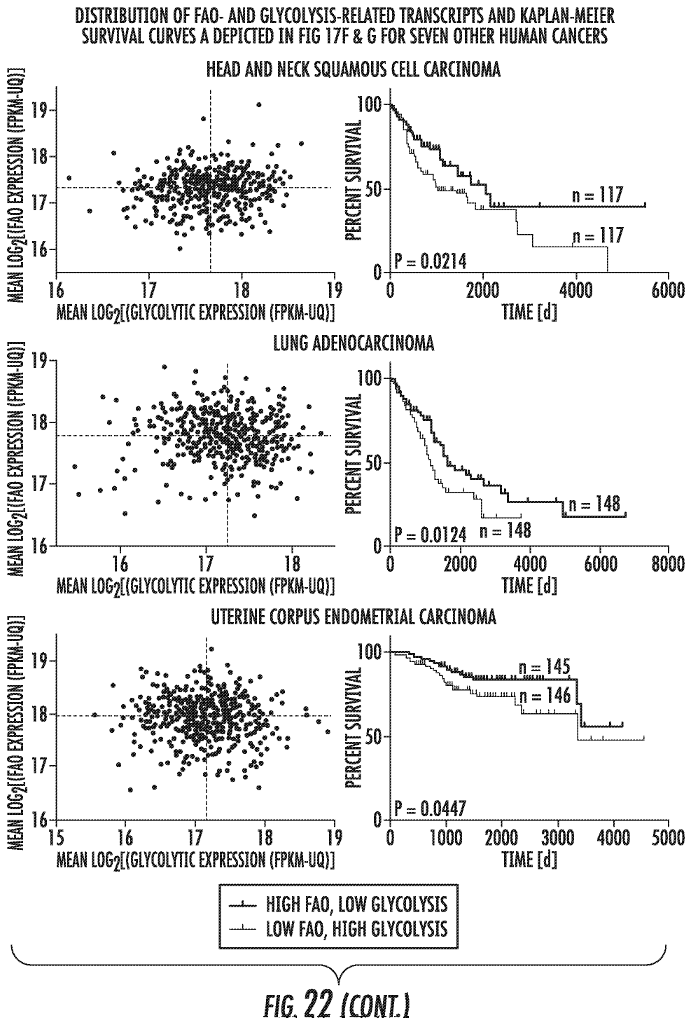

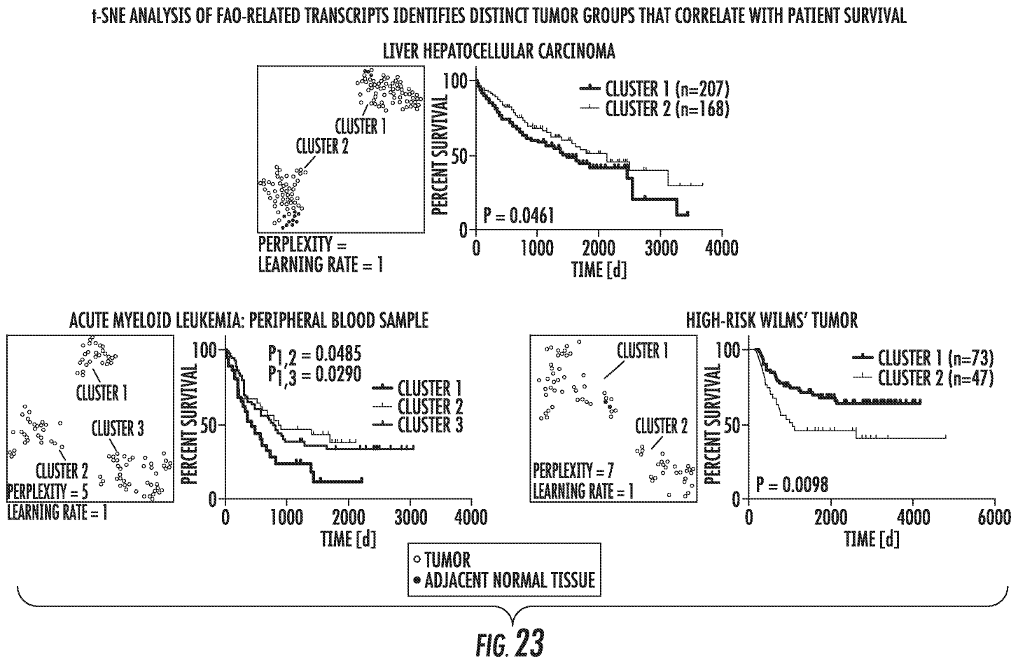

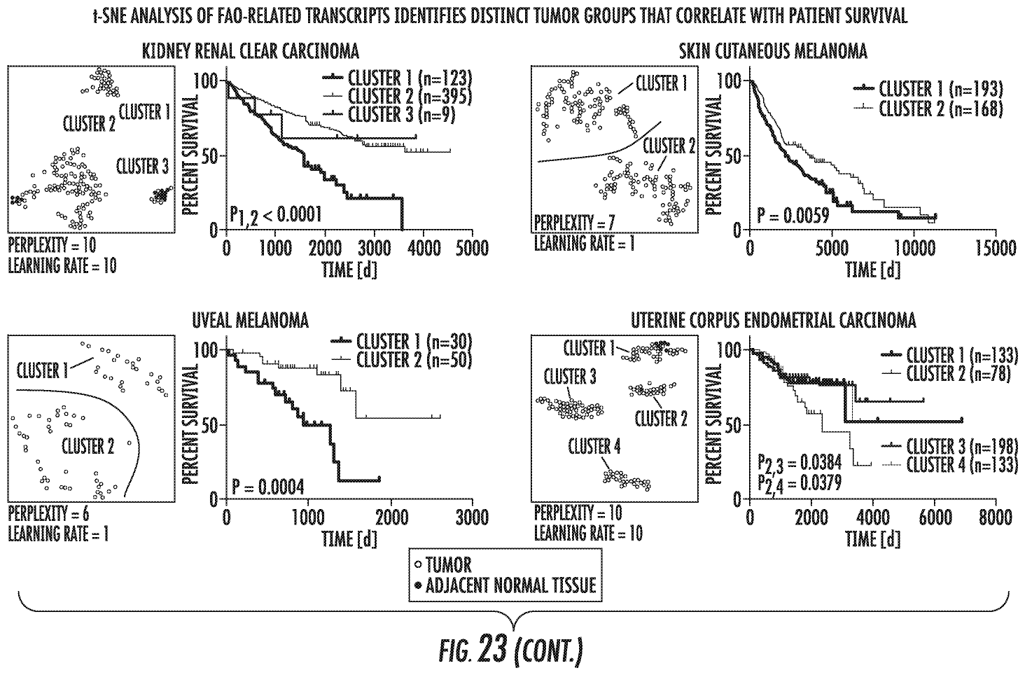

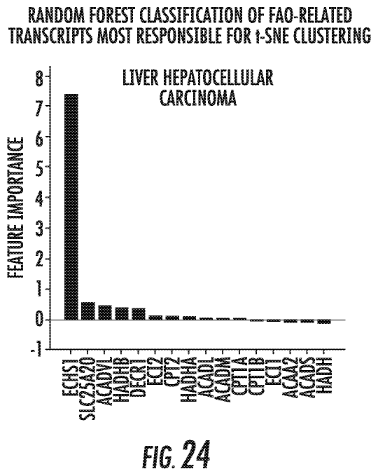

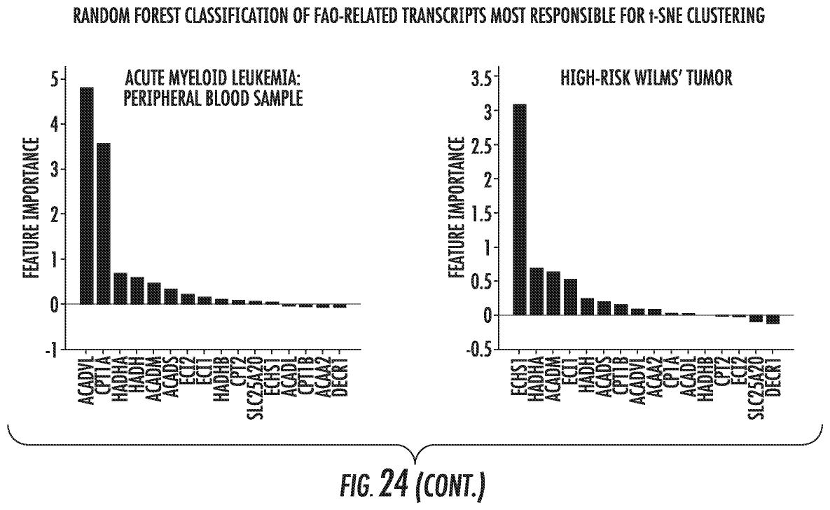

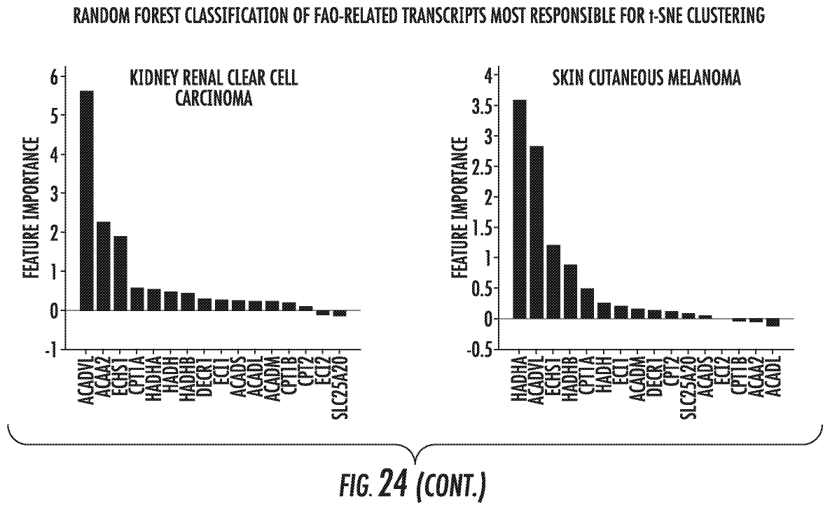

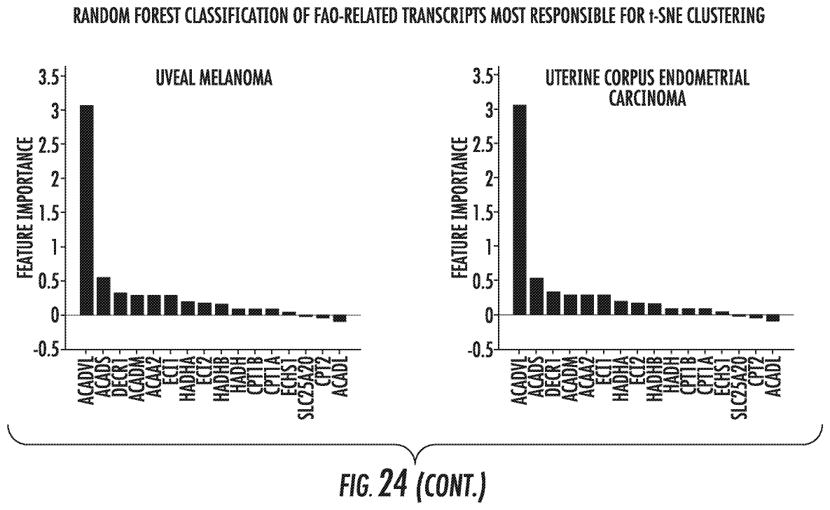

At 1603, global transcript expression patterns or profiles for tumors in the database are determined based on the RNA expression data for the tumors received at step 1601. In some implementations, the global transcript expression profiles are global cholesterol biosynthesis transcript expression profiles. In other implementations, the global transcript expression profiles are global FAO transcript expression profiles. This disclosure contemplates that the global transcript expression profiles can be global transcript expression profiles of other families of transcripts that have predictive value. At 1607, a global transcript expression profile (e.g., global cholesterol biosynthesis transcript expression profile and/or global FAO transcript expression profile) for the individual tumor sample is determined based on the RNA expression data received at step 1605. This disclosure contemplates that the global transcript expression patterns or profiles can be determined using a computing device (e.g., computing device 200 of FIG. 2). This can include a pre-processing step of calculating a respective relative expression for each of a plurality of enzymes involved in cholesterol biosynthesis and/or each of a plurality of FAO-related proteins. Pre-processing is performed on the raw RNA expression data received at steps 1601 (for the database of tumors) and 1605 (for the individual tumor sample). As described herein, a respective relative expression can be defined as a percentage contribution of an individual transcript to the total expression of the plurality of transcripts. After calculating the respective relative expression for each of a plurality of cholesterol biosynthesis transcripts or each of a plurality of FAO transcripts, a machine learning model is used to identify patterns of relative expression in the database of tumors while analyzing linear and non-linear relationships among the respective relative expression for each of the plurality of transcripts. As described herein, the machine learning model can optionally be t-SNE. The results of t-SNE analysis of cholesterol biosynthesis-related transcripts patterns are shown in FIG. 20, and the results of t-SNE analysis of FAO-related transcripts patterns are shown in FIG. 23. It should be understood that t-SNE is only one example machine learning model. This disclosure contemplates that other machine learning models can be used with the bioinformatics methods described herein. Patterns of transcript expression in the tumors from the database which have been identified by a machine learning model can be compared to clinical information about the patients from which these tumors derive with standard statistical tests. Such statistical tests can include, but are not limited to, t-tests, Chi-square tests, and/or log-rank tests. Such clinical information can include, but is not limited to, tumor type, patient survival, treatment response, or tumor biomarkers. Patterns of transcript expression that significantly associate with clinical parameters can be identified. At 1609, the global transcript expression profile from the individual tumor sample can be compared to the aforementioned transcript expression patterns identified in the database. Optionally, as described herein, global transcript expression for the tumors in the database, as well the individual tumor sample, can be graphically displayed with clusters using a three-dimensional (3D) map. The transcripts most responsible for t-SNE clustering are shown in FIG. 21 (cholesterol biosynthesis) and FIG. 24 (FAO). It should be understood that this allows the user to visualize patterns in the data set.

At 1611, a diagnosis, prognosis, or treatment recommendation is provided based on the comparison between the global transcript expression profile of the individual tumor sample and the transcript expression patterns identified in the database. For example, at least one of a clinical parameter (e.g., survivability metric), a molecular marker, or a tumor phenotype can be provided. This disclosure contemplates that any of the aforementioned information can be provided using a computing device (e.g., computing device 200 of FIG. 2). The comparison between the individual patient sample and the database of tumors is performed with the use of a classifier model. As described herein, a classifier model can be used to identify histologic subtype, prognostic group, or other clinical parameters. In some implementations, the classifier model is an artificial neural network (ANN) or a logistic regression (LR) classifier. It should be understood that ANN and LR classifiers are only example classifier models. This disclosure contemplates that other classifier models can be used with the bioinformatics methods described herein. The classifier model can differentiate between different types of tumor tissue. Alternatively or additionally, the classifier model can differentiate between subtypes of the same tumor tissue (i.e., sub-classify a particular type of tumor). In other words, using the global transcript expression pattern for the sample, it is possible (e.g., by comparison with a data set) to a diagnosis, prognosis, or treatment recommendation.

As described herein, the classifier model can be constructed using respective global transcript expression patterns for a plurality of known tissues (e.g., a majority of known tissues). As discussed above, when using a neural network, reliability and predictability improve when trained with more data. For example, global transcript expression patterns can be obtained by pre-processing raw RNA-seq expression data and applying a machine learning model (e.g., t-SNE) as described above. RNA-seq expression data for known tissue can be obtained from databases including, but not limited to, The Cancer Genome Atlas (TCGA). The global transcript expression patterns for known tissues can be used to train the classifier model. It should be understood that such training improves performance of the classifier model. Alternatively or additionally, it is possible to graphically display (e.g., by generating volcano plots comparing transcript expression patterns) one or more of the global transcript expression patterns, which can provide a visual indication of patterns in the data set, to identify the tissue of origin.

As described herein, some tumors with high ratios of FAO-related:glycolysis related transcripts were associated with more prolonged survival than those with low ratios. It has also been shown in other human tumors that the expression patterns of transcripts encoding FAO-related proteins and enzymes involved in cholesterol biosynthesis were predictive of survival as well. In a large number of human cancers, the ratio of transcripts related to FAO and glycolysis was predictive of survival, as were the patterns of expression of transcripts encoding enzymes catalyzing FAO and cholesterol biosynthesis. For example, in large cohorts of multiple human cancer types, the ratio of FAO:glycolysis-related transcripts or the expression patterns of transcripts involved in cholesterol biosynthesis or FAO were predictive of survival.

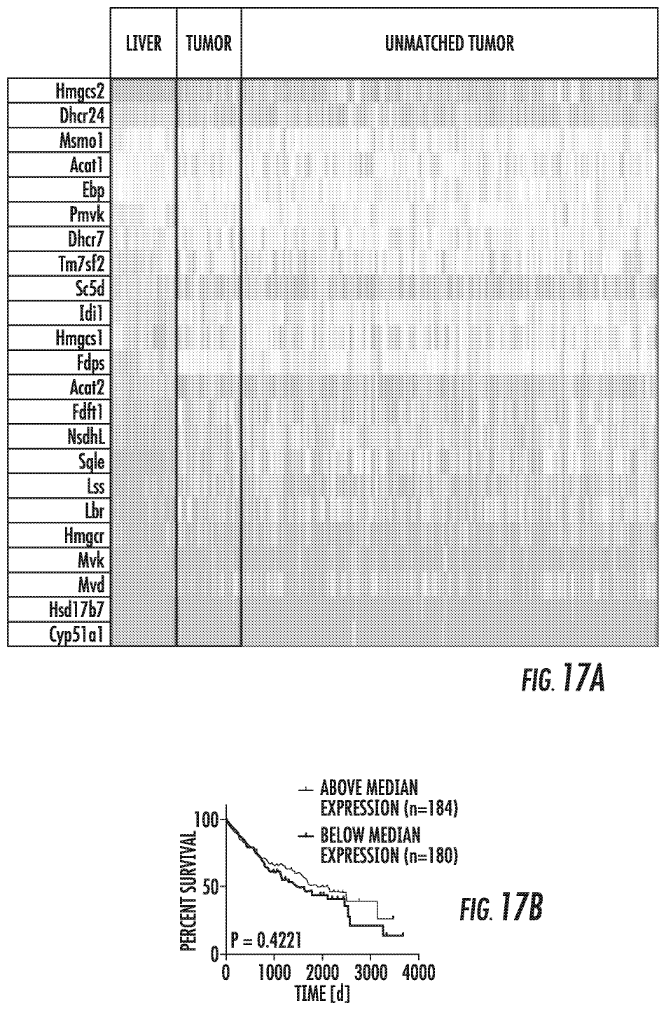

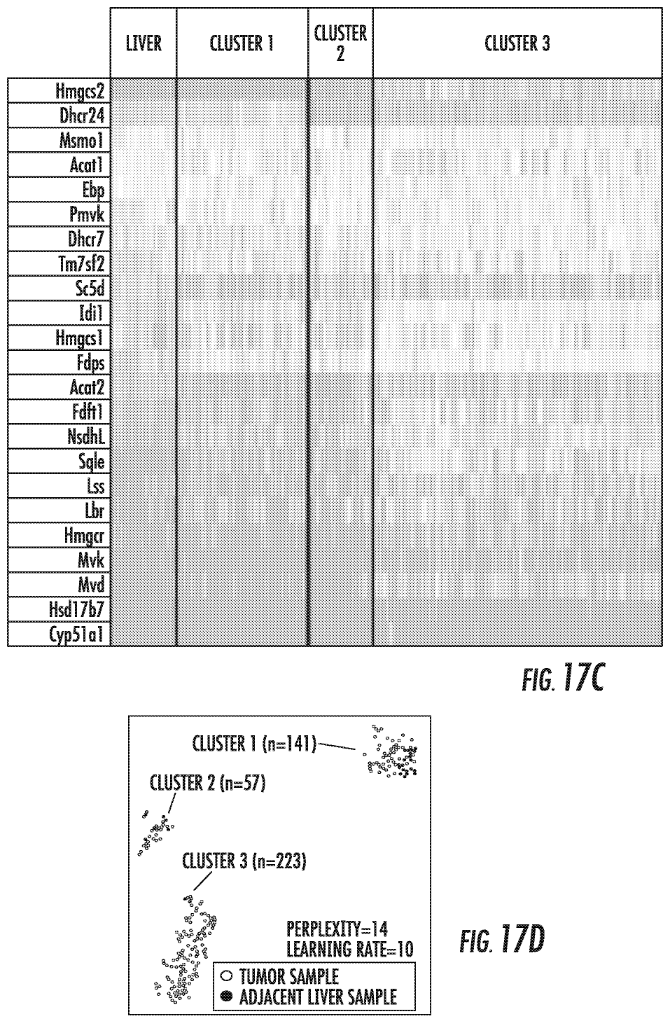

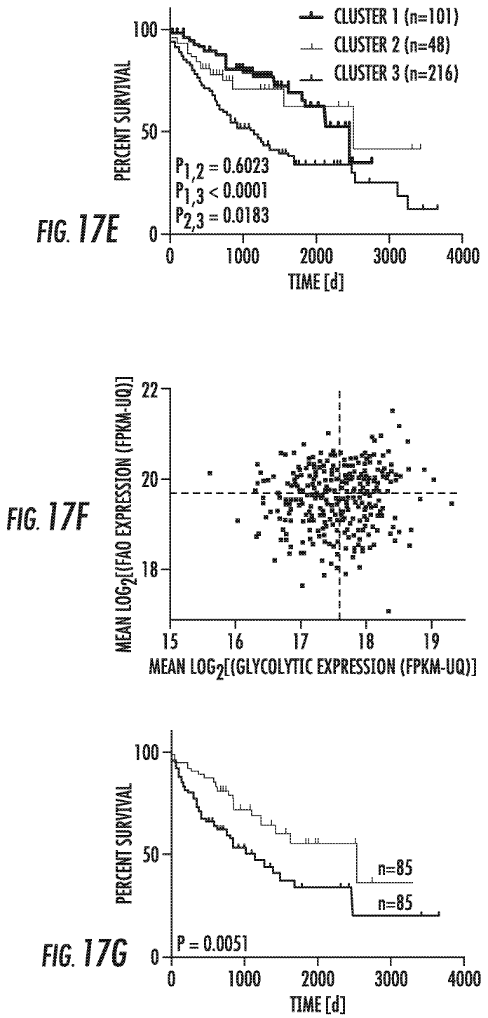

Transcripts involved in cholesterol biosynthesis, FAO and glycolysis predict patient survival. The mean expression levels of cholesterol biosynthetic enzyme-encoding transcripts (see FIG. 18A) did not significantly differ among 371 human HCC samples and 50 matched liver samples (see FIG. 17A) (average fold-differences between liver and tumor groups=1.042, P=0.54, paired ratio ttest) and the survival of patients whose tumors expressed the highest and lowest levels of these transcripts was similar (see FIG. 178). However, differences in transcript patterns were evident (see FIG. 17C), particularly when analyzed by t-SNE, a dimensionality reduction technique of particular utility for analyzing non-linear relationships. This identified three distinct HCC groups (see FIG. 17D) one of which was associated with a particularly unfavorable clinical course (see FIG. 17E). Eight other human tumor types were also identified whose patterns of cholesterol related transcript expression were similarly predictive of survival (see FIG. 20). A Random Forest Classification model showed that, in eight of the nine tumor cohorts, these patterns were largely determined by a small subset of transcripts, comprised of DHCR24, HMGCS2, PMVK and ACAT1/2 (see FIG. 21).

The same human TCGA data were next used to show that individuals whose HCCs were in the quadrant with the highest FAO:glycolytic transcript ratios (see FIG. 17F) survived longer relative to those with ratios in the lowest quadrant (see FIG. 17G). Similar survival differences were noted in seven other disparate tumor groups (see FIG. 22).

Like those for cholesterol biosynthesis, FAO transcript expression patterns were also found to be predictive of survival in HCC and six other cancers (see FIG. 23). Random Forest Classification again identified a small number of transcripts, particularly those for Acadv1 and Echs1 to be the primary determinants of pattern diversity (see FIG. 24).

Similar but less pronounced behaviors were seen with cholesterol synthesis-related transcripts (see FIG. 18B). Absolute levels of these also did not correlate with survival in a cohort of human HCC patterns (see FIG. 17B). However, their expression patterns did and extended to several other cancer types (see FIG. 20). These results were reminiscent of similar recent findings made with ribosomal protein transcripts (RPTs) in multiple cancers as described herein. The subset of cholesterol biosynthesis-related transcripts implicated as being the most responsible for the specific tumor patterns (see FIG. 21), namely DHCR24, HMGCS2 and PMVK, all have been previously shown to be deregulated in several different cancer types their individual levels have been shown to correlate with survival. The relationship between FAO and glycolysis in murine HCCs was also extended to multiple clinical cohorts. This showed that tumors with the highest ratios of FAO:glycolysis-related transcripts were associated with longer survival than those with the lowest ratios (see FIGS. 17F and 17G and FIG. 22).

### REFERENCES

1. Xue, S. & Barna, M. Specialized ribosomes: a new frontier in gene regulation and organismal biology. Nature reviews. Molecular cell biology 13, 355-369 (2012).
2. Noller, H. F., Hoffarth, V. & Zimniak, L. Unusual resistance of peptidyl transferase to protein extraction procedures. Science (New York, N.Y.) 256, 1416-1419 (1992).
3. Guimaraes, J. C. & Zavolan, M. Patterns of ribosomal protein expression specify normal and malignant human cells. Genome Biology 17 (2016).
4. Warner, J. R. & McIntosh, K. B. How common are extraribosomal functions of ribosomal proteins? Molecular cell 34, 3-11 (2009).
5. Zhou, X., Uao, W. J., Liao, J. M., Uao, P. & Lu, H. Ribosomal proteins: functions beyond the ribosome. Journal of Molecular Cell Biology 7, 92-104 (2015).
6. Ruggero, D. & Shimamura, A. Marrow failure: a window into ribosome biology. Blood 124, 2784-2792 (2014).
7. Yelick, P. C. & Trainor, P. A. Ribosomopathies: Global process, tissue specific defects. Rare diseases (Austin, Tex.) 3, e1025185 (2015).
8. Russo, A. & Russo, G. Ribosomal Proteins Control or Bypass p53 during Nucleolar Stress. International Journal of Molecular Sciences 18 (2017).
9. Shenoy, N., et al. Alterations in the ribosomal machinery in cancer and hematologic disorders. Journal of Hematology & Oncology 5, 32 (2012).
10. Boultwood, J., Pellagatti, A. & Wainscoat, J. S. Haploinsufficiency of ribosomal proteins and p53 activation in anemia: Diamond-Blackfan anemia and the 5q− syndrome. Advances in biological regulation 52, 196-203 (2012).
11. Gazda, H. T., et al. Ribosomal Protein L5 and L11 Mutations Are Associated with Cleft Palate and Abnormal Thumbs in Diamond-Blackfan Anemia Patients. American Journal of Human Genetics 83, 769-780 (2008).
12. Hong, M., Kim, H. & Kim, I. Ribosomal protein L19 overexpression activates the unfolded protein response and sensitizes MCF7 breast cancer cells to endoplasmic reticulum stress-induced cell death. Biochemical and biophysical research communications 450, 673-678 (2014).
13. Lai, M. D. & Xu, J. Ribosomal Proteins and Colorectal Cancer. Current genomics 8, 43-49 (2007).
14. Jung, Y., et al. Clinical validation of colorectal cancer biomarkers identified from bioinformatics analysis of public expression data. Clinical cancer research: an official journal of the American Association for Cancer Research 17, 700-709 (2011).
15. Yong, W. H., et al. Ribosomal Proteins RPS11 and RPS20, Two Stress-Response Markers of Glioblastoma Stem Cells, Are Novel Predictors of Poor Prognosis in Glioblastoma Patients. PloS one 10, e0141334 (2015).
16. Artero-Castro, A., et al. Expression of the ribosomal proteins Rplp0, Rplp1, and Rplp2 in gynecologic tumors. Human pathology 42, 194-203 (2011).
17. Paquet, E. R., et al. Low level of the X-linked ribosomal protein S4 in human urothelial carcinomas is associated with a poor prognosis. Biomarkers in medicine 9, 187-197 (2015).
18. Russo, A., Saide, A., Smaldone, S., Faraonio, R. & Russo, G. Role of uL3 in Multidrug Resistance in p53-Mutated Lung Cancer Cells. International Journal of Molecular Sciences 18 (2017).
19. Russo, A., et al. rpL3 promotes the apoptosis of p53 mutated lung cancer cells by down-regulating CBS and NFκB upon 5-FU treatment. Scientific reports 6 (2016).
20. Khan, F. H., et al. Acquired genetic alterations in tumor cells dictate the development of high-risk neuroblastoma and clinical outcomes. BMC Cancer 15 (2015).
21. Shi, C., Wang, Y., Guo, Y., Chen, Y. & Liu, N. Cooperative down-regulation of ribosomal protein L10 and NF-kappaB signaling pathway is responsible for the anti-proliferative effects by DMAPT in pancreatic cancer cells. Oncotarget 8, 35009-35018 (2017).
22. Fan, H., et al. Silencing of ribosomal protein L34 (RPL34) inhibits the proliferation and invasion of esophageal cancer cells. Oncology research (2017).
23. Kardos, G. R., Dai, M. S. & Robertson, G. P. Growth Inhibitory Effects of Large Subunit Ribosomal Proteins in Melanoma. Pigment cell & melanoma research 27, 801-812 (2014).
24. Sim, E. U., Chan, S. L., Ng, K. L, Lee, C. W. & Narayanan, K. Human Ribosomal Proteins RPeL27, RPeL43, and RPeL41 Are Upregulated in Nasopharyngeal Carcinoma Cell Lines. Disease markers 2016, U.S. Pat. No. 5,179,594 (2016).
25. Ajore, R., et al. Deletion of ribosomal protein genes is a common vulnerability in human cancer, especially in concert with TP53 mutations. EMBO molecular medicine 9, 498-507 (2017).
26. Goudarzi, K. M. & Lindstrom, M. S. Role of ribosomal protein mutations in tumor development (Review). International journal of oncology 48, 1313-1324 (2016).
27. Fancello, L., Kampen, K. R., Hofman, I. J., Verbeeck, J. & De Keersmaecker, K. The ribosomal protein gene RPL5 is a haploinsufficient tumor suppressor in multiple cancer types. Oncotarget 8, 14462-14478 (2017).
28. Naora, H., Takai, I., Adachi, M. & Naora, H. Altered cellular responses by varying expression of a ribosomal protein gene: sequential coordination of enhancement and suppression of ribosomal protein S3a gene expression induces apoptosis. The Journal of cell biology 141, 741-753 (1998).
29. van der Maaten, L. J. P. H., G. E. Visualizing High-Dimensional Data Using t-SNE. Journal of Machine Learning Research 9, 2579-2605 (2008).
30. De Keersmaecker, K. Ribosomopathies and the paradox of cellular hypo- to hyperproliferation. 125, 1377-1382 (2015).
31. Esposito, D., et al. Human rpL3 plays a crucial role in cell response to nucleolar stress induced by 5-FU and L-OHP. Oncotarget 5, 11737-11751 (2014).
32. Sun, X. X., Dai, M. S. & Lu, H. 5-fluorouracil activation of p53 involves an MDM2-ribosomal protein interaction. The Journal of biological chemistry 282, 8052-8059 (2007).
33. Sridhar, K., Ross, D. T., Tibshirani, R., Butte, A. J. & Greenberg, P. L. Relationship of differential gene expression profiles in CD34(+) myelodysplastic syndrome marrow cells to disease subtype and progression. Blood 114, 4847-4858 (2009).
34. Chaligné, R., et al. The inactive X chromosome is epigenetically unstable and transcriptionally labile in breast cancer. Genome Research 25, 488-503 (2015).
35. Spatz, A., Borg, C. & Feunteun, J. X-chromosome genetics and human cancer. Nature reviews. Cancer 4, 617-629 (2004).
36. Kobayashi, T., et al. Activation of the ribosomal protein L13 gene in human gastrointestinal cancer. International journal of molecular medicine 18, 161-170 (2006).
37. Hu, G., et al. MTDH Activation by 8q22 Genomic Gain Promotes Chemoresistance and Metastasis of Poor-Prognosis Breast Cancer. Cancer cell 15, 9-20 (2009).
38. Parris, T. Z, et al. Frequent MYC coamplification and DNA hypomethylation of multiple genes on 8q in 8p11-p12-amplified breast carcinomas. Oncogenesis 3, e95 (2014).
39. Taghavi, A., et al. Gene expression profiling of the 8q22-24 position in human breast cancer: TSPYL5, MTDH, ATAD2 and CCNE2 genes are implicated in oncogenesis, while WISP1 and EXT1 genes may predict a risk of metastasis. Oncology Letters 12, 3845-3855 (2016).
40. Ormandy, C. J., Musgrove, E. A., Hui, R., Daly, R. J. & Sutherland, R. L Cyclin D1, EMS1 and 11q13 amplification in breast cancer. Breast cancer research and treatment 78, 323-335 (2003).
41. Yuan, B. Z., Zhou, X., Zimonjic, D. B., Durkin, M. E. & Popescu, N. C. Amplification and overexpression of the EMS 1 oncogene, a possible prognostic marker, in human hepatocellular carcinoma. The Journal of molecular diagnostics: JMD 5, 48-53 (2003).
42. Barbashina, V., Salazar, P., Holland, E. C., Rosenblum, M. K. & Ladanyi, M. Allelic losses at 1p36 and 19q13 in gliomas: correlation with histologic classification, definition of a 150-kb minimal deleted region on 1p36, and evaluation of CAMTA1 as a candidate tumor suppressor gene. Clinical cancer research: an official journal of the American Association for Cancer Research 11, 1119-1128 (2005).
43. Vogazianou, A. P., et al. Distinct patterns of 1p and 19q alterations identify subtypes of human gliomas that have different prognoses( ). Neuro-Oncology 12, 664-678 (2010).
44. Horos, R., et al. Ribosomal deficiencies in Diamond-Blackfan anemia impair translation of transcripts essential for differentiation of murine and human erythroblasts. Blood 119, 262-272 (2012).
45. Landry, D. M., Hertz, M. I. & Thompson, S. R. RPS25 is essential for translation initiation by the Dicistroviridae and hepatitis C viral IRESs. Genes & Development 23, 2753-2764 (2009).
46. Muhs, M., et al. Structural basis for the binding of IRES RNAs to the head of the ribosomal 40S subunit. Nucleic acids research 39, 5264-5275 (2011).
47. Bellodi, C., et al. Loss of function of the tumor suppressor DKC1 perturbs p27 translation control and contributes to pituitary tumorigenesis. Cancer research 70, 6026-6035 (2010).
48. Chen, G., et al. Discordant protein and mRNA expression in lung adenocarcinomas. Molecular & cellular proteomics: MCP 1, 304-313 (2002).
49. Koussounadis, A., Langdon, S. P., Um, I. H., Harrison, D. J. & Smith, V. A. Relationship between differentially expressed mRNA and mRNA-protein correlations in a xenograft model system. Scientific reports 5, 10775 (2015).
50. Tian, Q., et al. Integrated genomic and proteomic analyses of gene expression in Mammalian cells. Molecular & cellular proteomics: MCP 3, 960-969 (2004).
51. Dreiseitl, S. & Ohno-Machado, L. Logistic regression and artificial neural network classification models: a methodology review. Journal of biomedical informatics 35, 352-359 (2002).

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A method of bioinformatics, comprising:
receiving RNA expression data for a sample of tumor;
determining a global ribosomal protein transcript (RPT) expression profile for the sample based on the RNA expression data; and
identifying a tissue of origin for the sample based on the global RPT expression profile for the sample.

2. The method of claim 1, wherein determining a global ribosomal protein transcript (RPT) expression profile for the sample comprises calculating a respective relative expression for each of a plurality of RPTs.

3. The method of claim 2, wherein the plurality of RPTs comprise RPTs for approximately eighty ribosomal proteins (RPs).

4. The method of claim 2, wherein a respective relative expression comprises a percentage contribution of an individual RPT to the total expression of the plurality of RPTs.

5. The method of claim 1, wherein identifying a tissue of origin for the sample comprises using a classifier model.

6. The method of claim 5, wherein the classifier model differentiates tumor tissue from normal tissue.

7. The method of claim 5, wherein the classifier model differentiates between different types of tumor tissue.

8. The method of claim 5, wherein the classifier model differentiates between subtypes of the same tumor tissue.

9. The method of claim 5, further comprising constructing the classifier model using respective global RPT expression profiles for a plurality of known tissues.

10. The method of claim 9, wherein identifying a tissue of origin for the sample comprises comparing quantitative differences between the global RPT expression profile for the sample and one or more of the respective global RPT expression profiles for the known tissues.

11. The method of claim 1, wherein the tissue of origin for the sample is identified based on dysregulation of the relative expression of one or more ribosomal proteins (RPs).

12. The method of claim 11, wherein the RPs comprise one or more of RPL3, RPL5, RPL8, RPL13, RPL30, RPL36, RPL38, RPL13, RPS4X, or RPS20.

13. The method of claim 1, further comprising providing a diagnosis, prognosis, or treatment recommendation based on the tissue of origin for the sample.

14. The method of claim 13, wherein providing a diagnosis, prognosis, or treatment recommendation comprises providing at least one of a clinical parameter, a molecular marker, or a tumor phenotype.

15. The method of claim 13, further comprising sub-classifying the tissue of origin for the sample based on the global RPT expression profile for the sample.

16. The method of claim 15, wherein the diagnosis, prognosis, or treatment recommendation is provided based on a sub-class of the tissue of origin for the sample.

17. The method of claim 1, further comprising:
receiving the sample of tumor;
extracting RNA from the sample;
isolating a plurality of RPTs from the extracted RNA; and
obtaining the RNA expression data from the isolated RPTs.

18. The method of claim 1, wherein the RNA expression data comprises RNA-seq data.

19. The method of claim 1, wherein the RNA expression data comprises microarray data.

20. The method of claim 1, wherein the tumor is an undifferentiated tumor.

21. The method of claim 1, further comprising:
receiving respective RNA expression data and respective clinical information for each of a plurality of tumors from a database;
determining respective global RPT expression profiles for the tumors in the database based on the respective RNA expression data;
identifying recurring patterns of RPT expression among the tumors in the database; and
comparing the recurring patterns of RPT expression with the respective clinical parameters.

22. The method of claim 21, wherein identifying a tissue of origin for the sample comprises comparing the global RPT expression profile for the sample to the respective global RPT expression profiles for the tumors in the database.

23. The method of claim 21, wherein identifying recurring patterns of RPT expression among tumors in the database further comprises applying a machine learning model that analyzes linear and non-linear relationships among the respective relative expression for each of the plurality of RPTs.

24. The method of claim 23, wherein the machine learning model is t-distributed stochastic neighbor embedding (t-SNE).

25. The method of claim 24, further comprising graphically displaying the global RPT expression pattern for the sample with clusters using a three-dimensional (3D) map.

26. A method of bioinformatics, comprising:
determining a global ribosomal protein transcript (RPT) expression profile for a sample of tumor, and
identifying a tissue of origin for the sample based on the global RPT expression pattern for the sample.

27. A method of bioinformatics, comprising:
receiving RNA expression data for a sample of tumor;
determining a global ribosomal protein transcript (RPT) expression profile for the sample based on the RNA expression data; and
providing a diagnosis, prognosis, or treatment recommendation based on the global RPT expression profile.

28. The method of claim 27, wherein providing a diagnosis, prognosis, or treatment recommendation comprises providing at least one of a clinical parameter, a molecular marker, or a tumor phenotype.

29. A method of bioinformatics, comprising:
receiving RNA expression data for a sample of tumor;
determining a global cholesterol biosynthesis transcript expression profile for the sample based on the RNA expression data; and
providing a diagnosis, prognosis, or treatment recommendation based on the cholesterol biosynthesis transcript expression profile.

30-39. (canceled)

40. A method of bioinformatics, comprising:
receiving RNA expression data for a sample of tumor;
determining a global fatty acid oxidation (FAO) transcript expression profile for the sample based on the RNA expression data; and
providing a diagnosis, prognosis, or treatment recommendation based on the FAO transcript expression profile.

41-48. (canceled)
